(19) United States
(12) Patent Application Publication
(10) Pub. No.: US 2011/0196494 A1
(43) Pub. Date: Aug. 11, 2011

(54) PERCUTANEOUS INTERBODY SPINE FUSION DEVICES, NUCLEAR SUPPORT DEVICE, SPINE FRACTURE SUPPORT DEVICE, DELIVERY TOOLS, PERCUTANEOUS OFF-ANGLE BONE STAPLING/NAILING FIXATION DEVICE AND METHODS OF USE

(75) Inventors: Joseph W. Yedlicka, Indianapolis, IN (US); Robert A. Till, JR., Avon, IN (US)

(73) Assignee: Osteo Innovations LLC, Indianapolis, IN (US)

(21) Appl. No.: 12/959,587

(22) Filed: Dec. 3, 2010

Related U.S. Application Data
(60) Provisional application No. 61/266,620, filed on Dec. 4, 2009.

(51) Int. Cl. A61F 2/44

(52) U.S. Cl. 623/17.16

(57) ABSTRACT

Percutaneous interbody spine fusion devices are provided. These devices may have a number of different designs and exemplary features. One device consists of a single rotating hollow cam cage with perforations (with or without fixation anchors) and a delivery tool. Another device consists of a counter-rotating cam cage (with or without fixation anchors) and a delivery tool. A third device consists of an expanding cam with anchors and delivery tool; this device may consist of a single expanding cam or a series of expanding cams. A delivery tool is included. A fourth device consists of a spring cage; this device may be a stand-alone device, can be combined with expanding cam device, and may be incorporated into a cage. A delivery tool is included. This spring cage may or may not have fixation anchors. A fifth device consists of a random coil support device that can be used as a nuclear or spine fracture support device; a delivery tool is included. A sixth device consists of a directional ribbon strip coil device and delivery tool. Also provided is a percutaneous off-angle bone stapling/nailing fixation device.

### REFERENCE TO RELATED APPLICATIONS

This application claims priority under 35 U.S.C. 119(e) to U.S. Provisional Patent Application No. 61/266,620, filed Dec. 4, 2009, the contents of which are herein incorporated by reference in its entirety.

### FIELD OF THE INVENTION

The present inventions relate to methods and devices for percutaneous spinal stabilization and fusion, and particularly stabilization and fusion of the interbody (intervertebral body) space. These inventions also relate to nuclear and vertebral fracture support devices and methods.

### BACKGROUND OF THE INVENTION

The individual vertebrae in the spine are joined to each other at three sites; the fibrocartilaginous intervertebral disc and two facet joints. Each vertebra has an articulating surface (facet) on the left and right sides; when joined with the articulating surfaces (facets) of the adjacent vertebrae, these articulating surfaces form facet joints. The vertebral bodies of the individual vertebrae are separated by intervertebral discs formed of a tough outer fibrous cartilage ring enclosing a central mass “jelly-like” semi-fluid mass, the nucleus pulposus that provides for cushioning and dampening of compressive forces to the spinal column. The adjacent surfaces of the vertebral bodies that abut the discs are covered with thin layers of hyaline cartilage. Several ligaments (supraspinous, interspinous, anterior and posterior longitudinal, and the ligamentum flavum) hold the vertebrae in position yet permit a limited degree of movement. The vertebral bodies are located anteriorly and together with the intervertebral discs provide the majority of the weight bearing support of the vertebral column. Each vertebral body has relatively strong cortical bone comprising the outer surface of the body and weak bone (cancellous) comprising the central portion of the vertebral body.

Persistent, chronic low back pain is often secondary to degeneration of the lumbar discs. With advancing age and degenerative disease, the water content of the nucleus pulposus diminishes and is replaced by fibrocartilage. The discs often lose height and become less elastic, the loss of disc height often results in bone spur formation, foraminal stenosis, canal stenosis, and resultant pain. In the spine, the pain can be treated by fusing the three sites of articulation: the intervertebral (interbody) space and the two facet joints.

There are two possible mechanisms that result in pain from diseased discs. The first theory is that the disc itself produces pain through trauma or degeneration and that removal of the disc is necessary to relieve the back pain. Typical surgeries to remove the disc and fuse the adjacent vertebrae together are performed in an open fashion and often involve extensive surgical manipulations with stripping and damaging of the paraspinal musculature. One method involves removing and replacing the disc with bone plugs and/or cages. These surgeries can also involve manipulations in the spinal canal itself. Other procedures include a variety of open lumbar fusion surgeries, with the anterior lumbar fusion often being performed as a “stand-alone” procedure.

The second theory is that the disc narrowing and degeneration leads to stress on all of the adjacent vertebral structures (including the vertebral bodies, ligaments, and facet joints). A number of devices and techniques involve implantation of spinal implants to reinforce or replace removed discs and to mechanically immobilize areas of the spine assisting in the eventual fusion of the treated adjacent vertebrae. One technique involves the use of pedicle screws and rods to immobilize the posterior aspect of the spine. Another technique involves the placement of anterior plate systems. A number of disc shaped replacements or artificial disc implants are also used. A type of disc reinforcement or augmentation implant is a hollow cylindrical cage that is placed in the interbody space after much of the disc material has been removed. These cages are typically placed in extensive open surgical procedures with considerable perioperative morbidity.

Another relatively common cause of back pain is spondylolysis. This disorder results from defects in the pars interarticularis which may be congenital or acquired. Spondylolysis can result in spondylolithesis (subluxation) of one vertebra on another. This subluxation can cause back and lower extremity pain from spinal canal stenosis and/or foraminal stenosis. There is a need for a percutaneous treatment device that can reduce the subluxation and prevent it from subluxing after the reduction.

Also, there are >700,000 vertebral body compression fractures/year in the United States, mainly in patients with osteoporosis. A number of devices and procedures are currently performed for treatment; however, an ideal procedure has not yet been developed.

It is also evident that there is a need for a percutaneous, off-angle, bone stapling/nailing fixation device to assist in orthopedic/neurosurgical procedures.

In summary, fusion of the intervertebral space has traditionally required open surgery. Unfortunately, these surgical procedures are extensive, often resulting in considerable peri-operative morbidity and prolonged recovery times. Various methods of fusing the intervertebral disc space have included surgical placement of cage devices, external plating and screws and transacral screw fixation. Most of the commonly used procedures require open surgery with resultant prolonged post-procedure recovery as well as morbidity and mortality associated with major surgery. Transacral screw fixation is only able to treat the lowest two lumbar levels.

Recently, there has been considerable, increasing interest in percutaneously placing a support device in the nucleus pulposus without removing the annular support fibers in patients with discogenic pain.

Also, there are a number of procedure and devices for treating vertebral body compression fractures. Some of these involve placing bone cement alone, another creates a cavity with a balloon and then places bone cement, another stacks wafers and surrounds the wafers with bone cement, and another places a containment bag filled with bone chips.

It is evident that there is a need for percutaneous devices, instrumentation, and techniques that result in safe, effective fusion and stabilization of the intervertebral (interbody) space. Also, there is a need for a percutaneous nuclear support device and delivery system and an improved, percutaneous vertebral body fracture support device and delivery system. Finally, there is a need for a percutaneous, off-angle bone stapling/nailing device to assist in orthopedic and neurosurgical procedures.

### SUMMARY OF THE INVENTION

The devices and methods disclosed herein relate to percutaneously placed interbody fusion devices, nuclear and vertebral body support devices; and their accompanying delivery tools and their methods of use.

1) A single rotating cam cage is described. The cam is oblong/eccentric in shape, allowing it to be placed in a flat dimension and then, once placed in the interbody space, rotated to secure it in place and also to provide lift to the interbody space. The single rotating cam cage has a number of fenestrations along its length. Bone graft material is meant to be placed into the central portion of this rotating fenestrated cam allowing for bony fusion. The length, height, and width of this cam can vary as appropriate for the interbody space. This rotating cam cage may also have fixation anchors integrated into the external body of the cam cage which protrude from the body and have pointed ends to provide additional fixation and immobility of the cam once deployed. The rotating cam cage may be constructed as a tapered or “stepped” device (thicker posteriorly) to aid in posterior elevation and lift; this aids in indirect decompression of spinal canal and neural foraminal stenoses. In addition, this device (especially with fixation anchors) can be used as a reduction device for spondylolithesis (subluxation). By placing this device(s) in a more horizontal fashion, it can result in the fixation anchors being able to move one vertebral body with respect to the adjacent vertebral body, improving alignment and helping to reduce subluxation (spondylolithesis). With either the cam shape itself wedged into the bone, or the rotating cam with anchors wedged into the bone, immediate mechanical interbody fixation can be achieved; the addition of bone graft allows for long-term bony fusion. A unique delivery tool for percutaneously delivering the rotating cam cage to the spine, comprising a delivery sheath and rotating (turning) member, is also described. The delivery tool engages with a delivery tool engagement feature in the cam to rotate the cam cage. If considered necessary, the cam can be further anchored into the endplates using the percutaneous, off-angle bone stapling/nailing device. Both the delivery tool and the cam cage may be cannulated for insertion over a guide pin or wire.

2) A Counter-rotating cam cage is described. This cam consists of two (or more) oblong/eccentric single rotating cams connected in series with swivel joints between the individual cams. The counter-rotating cam cage may have fixation anchors oriented in opposite directions which are integrated into the external body of the cam cage and protrude from the body having pointed ends. The counter-rotating cam also has multiple fenestrations along its length. Bone graft material is meant to be placed into the central portion of this fenestrated cam allowing for bony fusion. The length, height, and width of this counter-rotating cam cage can vary as appropriate for the interbody space. The counter-rotating cam cage may be constructed as a tapered or “stepped” device (thicker posteriorly) to aid in posterior elevation and lift; this aids in indirect decompression of spinal canal and neural foraminal stenoses. The counter-rotating cam cage has a unique delivery tool used through a delivery sheath for percutaneously delivering the counter-rotating cam cage to the spine. The delivery tool engages with a delivery tool engagement features located in the cam cages. Both the delivery tool and the cam cage may be cannulated for insertion over a guide pin or wire. The delivery tool allows the individual cams to be rotated (turned) in opposite directions, thus allowing for improved fixation with the integrated fixation anchors. The integrated fixation anchors are therefore “swiveled” in opposite directions, this results in opposing anchor fixation and aids in immediate interbody fixation. When bone graft material is added to the device, the device anchors result in immediate mechanical interbody fixation as well as long-term bony fusion. This device may be placed with hand-turning device or a power device such as an impact wrench. If considered necessary, this device can be further anchored into the endplates using the percutaneous off-angle bone stapling/nailing device.

3) An expanding cam is described. This device consists of side-by-side or two integrated cams meant to open in opposite directions, a pivot pin , an anchor rod comprising a mating hole and a threaded surface opposite the mating hole, and a locking nut comprising an integral washer and an interior threaded surface. Each cam comprising two pin holes, a cam surface and one or more protrusions extending from cam surfaces, the protrusions having pointed ends (i.e., anchoring devices). The pin holes of each cam are coupled to the mating hole of the anchor rod via the pivot pin and anchor rod is coupled to the locking nut via their threaded surfaces, and wherein the cams are rotated 180 degrees relative to each other when assembled. The anchor devices extending from the cams are meant to fix the individual cams into the cortical vertebral body endplates providing for mechanical fixation and lift. The oblong/eccentric cam shapes of the individual cam elements also provide for fixation and lift. This expanding cam can also be constructed in series with two (or more) expanding cams which can all be rotated to provide mechanical fixation and lift. If constructed in series, the posterior device may be constructed with additional height to aid in additional posterior elevation and lift. This expanding cam allows for immediate mechanical interbody fixation and motion prevention; placement of multiple expanding cams (e.g. two on each side of the vertebra) allows for multi-point fixation, the operator is also able to control posterior “lift” by placing slightly larger expanding cams posteriorly. A unique delivery tool configured for percutaneously delivering the expanding cam assembly to the spine is also described. Both the delivery tool and expanding cam assembly may be cannulated for insertion over a guide pin or wire.

4) A spring cage is described. The spring cage has a helical spring body having an inner and an outer diameter, a cross section diameter, a defined pitch length and a defined number of turns. The cross section may be circular or non-circular in shape. The inner and outer diameters may be uniform or variable along the length of the spring body, such that the external contour of the spring body is non-cylindrical or tapered. This spring-like device is inserted through a small delivery tool which then expands automatically when deployed. This spring cage can be placed as a “stand-alone” device in the nuclear space to provide support, lift, and recoil flexibility. One or more of these devices can be placed in the nuclear space. The ends of the spring cage may or may not have anchor devices for additional fixation.

A variation of the spring cage is described. The spring cage may also be made of a double or triple interweaved spring design formed by disposing one or more additional spring cages within the interior of the spring cage. The hands of the one or more additional spring cages may be in the same direction or opposite directions. This design meant to provide increased strength and support as well as recoil flexibility and also to provide smaller side openings to better contain bone graft material (meant to be placed into the central portion of this device to allow for bony fusion). The ends of the spring cage may or may not have anchor devices for additional fixation. Exemplary benefits of this spring cage include improved conformation to the adjacent vertebral end plates and the provision of inter-vertebral disc space flexible lift. The inherent flexibility of the spring itself allows for some motion preservation in the disc and/or nuclear space. The stiffness/flexibility of the spring cage can be adjusted depending on its intended use (nuclear support device or interbody fusion device). Also, this spring cage is delivered through an introducer smaller than the fully expanded cage, thus minimizing trauma to the disc space.

Another variation of the spring cage is described. The spring cage can be combined with one or more expanding cams to provide additional mechanical fixation and lift. A unique delivery tool for percutaneously delivering the spring cage to the spine is also provided. Both the delivery tool and the spring cage may be cannulated for insertion over a guide pin or wire.

Another fixation method for the spring cage is provided. A fixation staple anchor for the spring cage is described. This employs the percutaneous off-angle bone stapling/nailing device.

Another variation of the spring cage is described. The spring cage can be incorporated into an expandable, cylindrical shaped containment cage formed from a biocompatible material (e.g., PEEK polymer, stainless steel, titanium). The containment cage has two side walls having a proximal and a distal end and multiple perforations, end plates at the distal end of the side walls, and a plurality of bridging arms connecting the side walls. This spring cage/containment cage design would allow the spring cage to extrude through the openings in between the bridging arms in the containment cage to provide better fixation and also to provide for appropriate sized fenestrations to allow for bone graft containment and resultant bony fusion. An advantage of the spring cage incorporated into a containment cage is that it would better conform to the concave and often irregular surfaces of the adjacent vertebral endplates and provide recoil flexibility in addition to bone graft containment, fixation and lift. A unique delivery tool configured for percutaneously delivering the spring cage/containment cage to the spine is also provided. Both the delivery tool and the spring cage/containment cage assembly may be cannulated for insertion over a guide pin or wire.

Any of the above spring cages may be constructed as a tapered or “stepped” device (thicker posteriorly) to aid in posterior elevation and lift. The spring cages can also be constructed as thicker in the middle and tapered at the ends when used as a nuclear support device.

5) A Random Coil Support Device is described. This device consists of strips or coils of pre-formed metal or biocompatible material having a defined length, a cross section diameter, a distal end and a proximal end, the distal end having a blunted shape. The coil body is adapted to buckle along the length of the body when force is applied against the ends of the coil. The device is inserted into the spine through a small, unique delivery tool. Once inserted into the nuclear space, disc space, or vertebral body fracture, the pre-formed coils or strips would randomly open, providing support, lift, and recoil flexibility. A unique delivery tool configured for percutaneously delivering the random coil support device to the nuclear space, disc space, or vertebral body fracture is also provided. Both the delivery tool and the random coil support device may be cannulated for insertion over a guide pin or wire.

A variation of the Coil Support Device consists of a directional ribbon strip having a rectangular cross section and preformed bends along the length of the strip. The ribbon strip would collapse at the pre-formed bends providing directional force, support, and lift as well as some recoil flexibility. A unique delivery tool for percutaneously delivering the directional ribbon strip to the nuclear space, disc space, or vertebral body fracture is also provided. Both the delivery tool and the directional ribbon strip device may be cannulated for insertion over a guide pin or wire.

Any of the spinal devices described above can be formed from a biocompatible material, such as stainless steel, titanium, nitinol or PEEK polymer.

6) A Percutaneous Off-Angle Bone Stapling/Nailing Device is provided. The bone stapling/nailing device is comprised of a guide body assembly, a ram (driver), a cartridge, and the fixation device (e.g., staples, nails or brads). The guide body assembly is comprised of a rigid guide body, a flexible guide, and a cartridge adapter. The flexibility of the guide, which is curved to direct the cartridge radially, allows the distal end of the guide body assembly to deflect during insertion, allowing for off-angle fixation device placement and removal. This device is designed to percutaneously place curved staples, nails, brads, or other types of anchoring/fixation devices, to provide anchor fixation or bone union. Exemplary features of this off-angle, percutaneous staple/nail/brad placement device include a curved staple or nail or brad, various staple, nail or brad shapes (standard wire staple design, barbed points, brad points, metal side fletching anchors, etc), and a flexible staple neck, to allow for fixation devices to be deployed off-axis to the delivery tool. The staples, nails or brads can be in a cartridge (new staple, nail or brad snapped in each time) or the staple/nail/brad can be loaded through the end. The cartridge may have various configurations (e.g., single use, reloadable, multiple staples/nails/brads). There can be multiple staples, nails or brads (like a regular staple or nail gun). The percutaneous off-angle fixation staple/nail/brad anchor delivery tool driver can be driven forward with different driving forces: it can be tapped with a hammer (manual), hit with a single forcible blow (like a standard staple or nail gun), or hit multiple times with smaller blows (impact hammer). Alternatively, the driver can be power driven (pneumatic, electric, etc.) for single hard blow, or a powered impact hammer type device that generates a high repetition of smaller blows. The fixation staple anchor delivery tool may have a notch on its distal tip to locate and center over a device (e.g. wire coil of a spring cage). The off-angle design and small size allow the placement of fixation staples or nails at an angle different from the device placement direction into a bone. Thus, this allows “sideways” placement of staples or nails into a bone. The flexible neck of the delivery tool allows the end of the staple or nail cartridge to deflect radially to contact the spring cage wire; another deployment device can be added to help force the staple out of the delivery tool. If smaller staples are used, two staples can be deployed at the same time, 180 degrees opposed (one in each end plate). The fixation staple anchor and delivery tool can be made in various sizes and can be used for other bony neurologic, orthopedic, and interventional procedures.

### BRIEF DESCRIPTION OF THE DRAWINGS

The above-mentioned and other features and objects of this invention, and the manner of attaining them, will become more apparent and the invention itself will be better understood by reference to the following description of embodiments of the invention taken in conjunction with the accompanying drawings, where:

FIG. 1 is a top, side perspective view of the embodiment of an exemplary rotating cam cage fashioned in accordance with the principles of the present invention.

FIG. 2 is a top, side perspective view of an alternate embodiment of the rotating cam cage in FIG. 1 showing incorporated fixation features.

FIG. 3 is an end view of the rotating cam cage in FIG. 1 positioned between the top and bottom plates of two adjacent vertebrae.

FIG. 4 is an end view of the rotating cam cage in FIG. 1 positioned between the top and bottom plates of two adjacent vertebrae as in FIG. 3 rotated 90 degrees clockwise.

FIG. 5 is an end view of the rotating cam cage in FIG. 2 positioned between the top and bottom plates of two adjacent vertebrae rotated 90 degrees clockwise as in FIG. 3.

FIG. 6 is a top, side perspective view of the rotating cam cage in FIG. 1 with its delivery tool.

FIG. 7 is a top, side perspective exploded view of the rotating cam cage in FIG. 1 with its delivery tool.

FIG. 8 is a close-up top, side perspective exploded view of the distal end of the delivery tool and the rotating cam cage in FIG. 1.

FIG. 9 is a cross-section top, side perspective view of the distal end of the delivery tool and rotating cam cage in FIG. 1.

FIG. 10 is a top, side perspective view of the embodiment of an exemplary counter-rotating cam cage fashioned in accordance with the principles of the present invention.

FIG. 11 is a top, side exploded perspective view of the counter-rotating cam cage in FIG. 10.

FIG. 12 is a cross-section top, side perspective view of the counter-rotating cam cage in FIG. 10.

FIG. 13 is a top, side perspective view of the counter-rotating cam cage in FIG. 10 with its delivery tool.

FIG. 14 is a top, side exploded perspective view of the counter-rotating cam cage in FIG. 10 with its delivery tool.

FIG. 15 is an enlarged top, side exploded perspective view of the distal end of the counter-rotating cam cage in FIG. 10 with its delivery tool.

FIG. 16 is an enlarged side cross-sectional view of the distal end of the counter-rotating can cage in FIG. 10 with its delivery tool.

FIG. 17 is an enlarged top, side perspective view of the distal end of the counter-rotating cam cage in FIG. 10 with its delivery tool.

FIG. 18 is an enlarged top, side perspective view of the distal end of the counter-rotating cam cage in FIG. 10 with its delivery tool wherein the proximal rotating cam cage has been rotated 90 degrees relative to the distal rotating cam cage.

FIG. 19 is a top, rear perspective view of the counter-rotating cam cage in FIG. 10 with its delivery tool as it would be placed into the disk space during the procedure.

FIG. 20 is a top view of components shown in FIG. 19.

FIG. 21 is an enlarged top view of FIG. 20 with the top vertebrae and top half of the disk removed revealing the counter-rotating cam cage in place within the disk space.

FIG. 22 is a side view of an alternate embodiment of the rotating cam cage in FIG. 1 showing multiple fixation features and a tapered body.

FIG. 23 is a top, side perspective view of an alternate embodiment of the rotating cam cage in FIG. 1 showing multiple fixation features and a tapered body.

FIG. 24 is a top, side perspective view of the embodiment of an exemplary expanding cam fashioned in accordance with the principles of the present invention shown in its delivery position with a section of the sheath removed for clarity.

FIG. 25 is a top, side perspective view of the expanding cam in FIG. 24 shown exploded in its delivery position with a section of the sheath removed for clarity.

FIG. 26 is a top, side perspective cross-section view of the expanding cam in FIG. 24 shown in its delivery position.

FIG. 27 is a top, side perspective view of the expanding cam in FIG. 24 with the expanding cam extended from inside its delivery sheath and the nut driver retracted to reveal the nut.

FIG. 28 is a front, side perspective view of the expanding cam in FIG. 24 with the expanding cam extended from inside its delivery sheath and the nut driver retracted to reveal the nut.

FIG. 29 is a top, side perspective view of the delivery tool for the expanding cam shown in FIG. 24.

FIG. 30 is a top, side exploded perspective view of the delivery tool and the expanding cam shown in FIG. 24.

FIG. 31 is a top, side exploded perspective view of the expanding cam in FIG. 24.

FIG. 32 is a top, side perspective view of the expanding cam in FIG. 24 shown in a partially expanded position.

FIG. 33 is a top, side perspective view of the expanding cam in FIG. 24 shown in a fully expanded position.

FIG. 34 is a top, side perspective view of the expanding cam in FIG. 24 shown in a fully expanded position with the delivery tool removed.

FIG. 35 is a side view of the expanding cam in FIG. 24 in its fully collapsed position.

FIG. 36 is a side view of the expanding cam in FIG. 24 in partially expanded position.

FIG. 37 is a side view of the expanding cam in FIG. 24 in fully expanded position.

FIG. 38 is a top, side perspective view of the embodiment of an exemplary spring cage fashioned in accordance with the principles of the present invention.

FIG. 39 is a top view of 2 of the spring cages in FIG. 38 positioned within the disk space atop a vertebral body.

FIG. 40 is a side view of 2 of the spring cages in FIG. 38 positioned within the disk space between 2 vertebral bodies where the top half of the disk is removed for clarity.

FIG. 41 is a front view of 2 of the spring cages in FIG. 38 positioned within the disk space atop a vertebral body.

FIG. 42 is a top view of 2 of the spring cages in FIG. 38, one of which has been elongated, positioned in a different manner within the disk space atop a vertebral body.

FIG. 43 is a top, side perspective view of an alternate embodiment of the spring cage in FIG. 38 wherein a second spring cage has been positioned within the first.

FIG. 44 is a top, side perspective view of an alternate embodiment of the spring cage in FIG. 38 depicting a different cross-sectional shape for the wire that forms the spring cage.

FIG. 45 is a top, side perspective view of an alternate embodiment of the spring cage in FIG. 38 wherein the exterior profile has a varying contour.

FIG. 46 is a top, side perspective view of an alternate embodiment of the spring cage in FIG. 38 wherein the exterior profile has a tapered profile.

FIG. 47 is a top, side perspective view of the delivery tool for the spring cage shown in FIG. 38.

FIG. 48 is a top, side exploded perspective view of the delivery tool for the spring cage shown in FIG. 38.

FIG. 49 is an enlarged top, side perspective view of the distal end of the delivery tool for the spring cage shown in FIG. 38 with a section of the introducer tube removed for clarity.

FIG. 50 is an enlarged top, side perspective view of the distal end of the delivery tool for the spring cage shown in FIG. 38 with a section of the introducer tube removed for clarity showing partial deployment.

FIG. 51 is an enlarged top, side perspective view of the distal end of the delivery tool for the spring cage shown in FIG. 38 with a section of the introducer tube removed for clarity showing three-quarter deployment.

FIG. 52 is an enlarged top, side perspective view of the distal end of the delivery tool for the spring cage shown in FIG. 38 with a section of the introducer tube removed for clarity showing full deployment.

FIG. 53 is a top, side perspective view of the embodiment of an exemplary spring cage containment cage fashioned in accordance with the principles of the present invention.

FIG. 54 is an enlarged top, side perspective view of the distal end of the delivery tool for the spring cage shown in FIG. 38 and containment cage shown in FIG. 53 with a section of the introducer tube removed for clarity.

FIG. 55 is an enlarged top, side perspective view of the distal end of the delivery tool for the spring cage shown in FIG. 38 and containment cage shown in FIG. 53 with a section of the introducer tube removed for clarity showing full deployment.

FIG. 56 is a top, side perspective view of the embodiment of an exemplary random coil support device fashioned in accordance with the principles of the present invention.

FIG. 57 is a top, side perspective view of the delivery tool for the random coil support device shown in FIG. 56.

FIG. 58 is a top, side exploded perspective view of the delivery tool for the random coil support device shown in FIG. 56.

FIG. 59 is an enlarged top, side perspective view of the distal end of the deployment rod engaged with the proximal end of the random coil support device shown in FIG. 56.

FIG. 60 is a top, side exploded perspective view of the delivery tool for the random coil support device shown in FIG. 56 with the distal end of the random coil support device partial deployed.

FIG. 61 is a top, side perspective view of the delivery tool for the random coil support device shown in FIG. 56 positioned within the disk space between 2 vertebral bodies.

FIG. 62 is an enlarged top, side perspective view of the delivery tool for the random coil support device shown in FIG. 56 positioned within the disk space between 2 vertebral bodies.

FIG. 63 is an enlarged top, side perspective view of the delivery tool for the random coil support device shown in FIG. 56 positioned within the disk space between 2 vertebral bodies with the random coil support device partially deployed.

FIG. 64 is an enlarged top, side perspective view of the delivery tool for the random coil support device shown in FIG. 56 positioned within the disk space between 2 vertebral bodies with the random coil support device further deployed, coiling within the disk.

FIG. 65 is an enlarged top, side perspective view of the delivery tool for the random coil support device shown in FIG. 56 positioned within the disk space between 2 vertebral bodies with the random coil support device fully deployed, coiled within the disk

FIG. 66 is a top, side perspective view of an alternate embodiment of the random coil support device in FIG. 56, referred to as a flexible coil, wherein the cross section of the device is rectangular in shape with alternating bends in a single plane.

FIG. 67 is a top, side perspective view of the flexible coil support device shown in FIG. 66 partially collapsed.

FIG. 68 is a top, side perspective view of the flexible coil support device shown in FIG. 66 fully collapsed into its final position.

FIG. 69 is a top, side perspective view of the delivery tool for the flexible coil support device shown in FIG. 66.

FIG. 70 is a top, side perspective view of an alternate embodiment of the spring cage in FIG. 38 and the expanding cam in FIG. 24 wherein the two devices have been deployed together within the disk in two different configurations.

FIG. 71 is a top, side perspective view of the embodiment of an exemplary stapler used to anchor the spring cage shown in FIG. 38, fashioned in accordance with the principles of the present invention.

FIG. 72 is a top, side exploded perspective view of the stapler shown in FIG. 71.

FIG. 73 is an enlarger top, side exploded perspective view of the stapler shown in FIG. 71 highlighting the distal end.

FIG. 74 is a top, side perspective cross-section view of the distal end of the stapler shown in FIG. 71.

FIG. 75 is a top, side perspective view of the stapler shown in FIG. 71 positioned within the disk space relative to the spring cage shown in FIG. 38.

FIG. 76 is an enlarged top, side perspective view of the distal end of the stapler shown in FIG. 71 positioned within the disk space relative to the spring cage shown in FIG. 38

FIG. 77 is a side cross-sectional view of the stapler shown in FIG. 71 positioned within the disk space relative to the spring cage shown in FIG. 38 showing the various stages of deploying the staple.

FIG. 78 is a top, side cross-sectional perspective view of an alternate embodiment of the stapling tool cartridge wherein the staple is formed as a single curved nail.

### DETAILED DESCRIPTION

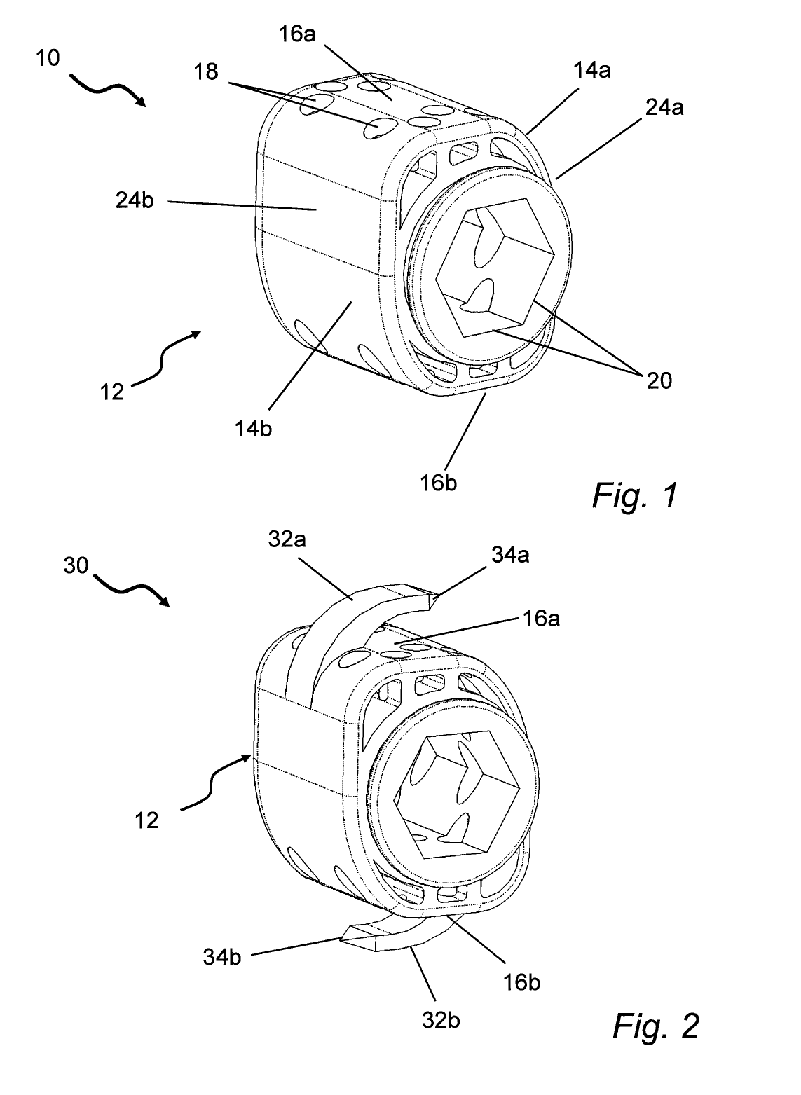

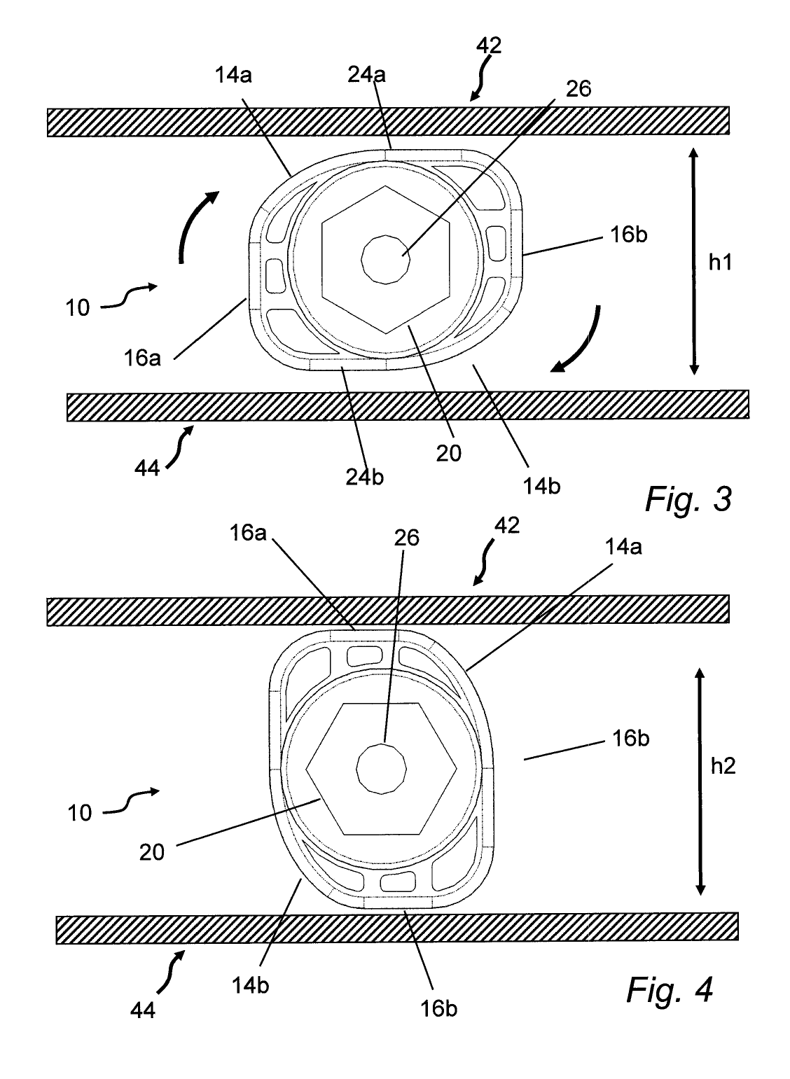

Referring to FIG. 1, there is depicted a rotating cam cage generally designated 10, fashioned in accordance with the present principles. The rotating cam cage consists of a single structure, cam body 12 which may be formed in various manners from an appropriate, biocompatible metal (such as stainless steel, titanium, etc.) or polymer (such as PEEK polymer). The exterior profile is shaped to create cam surfaces 14a and 14b that connect the base planar sides 24a and 24b with the expanded planar sides 16a and 16b. Referring to FIGS. 3 and 4, in use, the rotating cam cage is inserted between two adjacent vertebrae 42 and 44 with the base planar surface 24a and 24b parallel to the top and bottom plates of the vertebral bodies. The cam body 12 is then rotated 90 degrees clockwise to a position shown in FIG. 4. Rotation is accomplished using an delivery tool that engages the cam body 12 through features shown here as a typical hex opening 20. During rotation, the cam surfaces 14a and 14b engage the top and bottom plates of the adjacent vertebrae 42 and 44 causing them to separate from their initial height (h1 shown in FIG. 3) to their final height (h2 shown in FIG. 4). The cam body 12, in one variation, may for the most part be solid (excluding the delivery tool engagement feature 20). An alternative embodiment would create a mostly hollow cam body 12 (as shown in FIG. 1) that can be filled with bone graft material. In this configuration, fenestrations 18 of various sizes and cross section pass from the exterior of the cam body 12 to the interior, hollow volume. The fenestrations 18 would be position on the same sides of the cam body as the expanded planar surfaces 16a and 16b which are in contact with the bony plates of the vertebra 42 and 44 after rotation into final position. The length of the cam body 20 can vary to accommodate a single long cam or multiple, shorter cam placed with the disk.

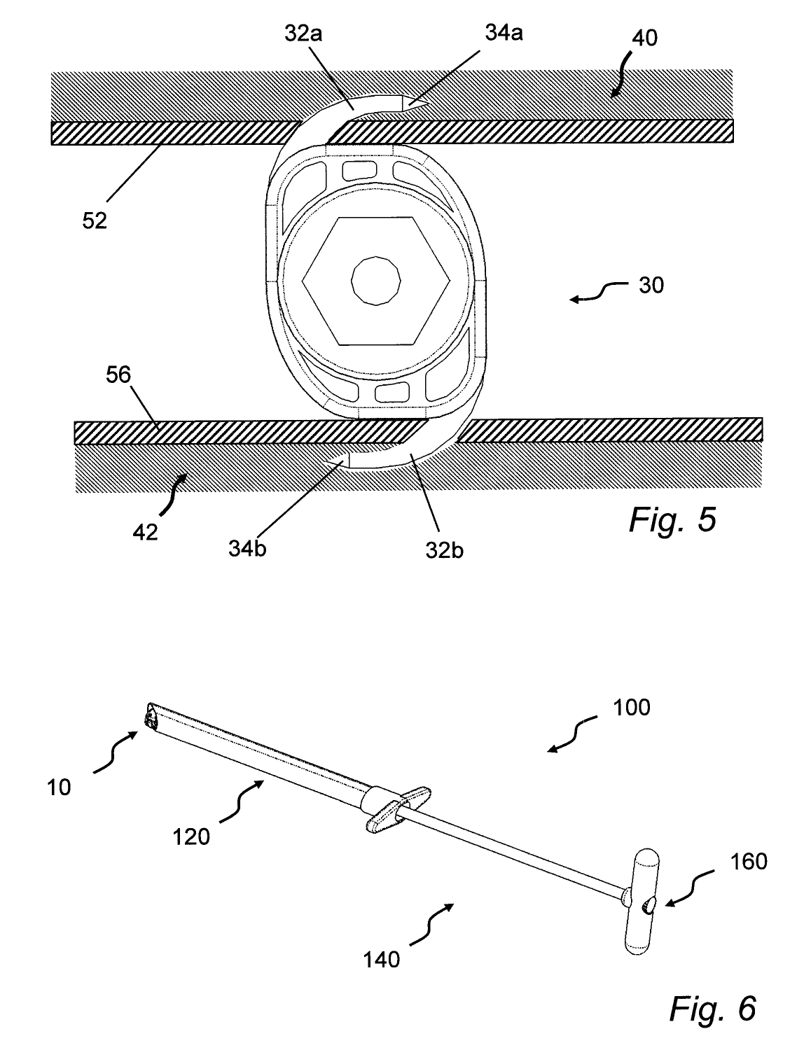

FIG. 2 shows an alternate embodiment of the rotating cam cage 30 that contains fixation anchors 32a and 32b. The anchors extend from the cam body 12 out over the expanded planar surfaces 16a and 16b.The ends of the anchors have a pointed edge 34a and 34b. Referring to FIG. 5, the pointed ends 34a and 34b of the fixation anchors 32a and 32b engage the boney plates of the vertebrae 40 and 42 as the cam 30 is rotated into position piercing through the outer cortical bone 52 and 56. This provides a structural fixation between the vertebrae 40/42 and cam 30. Note that, though shown here as a single structure on either side, there could exist, multiple fixation anchors of various designs on each end.

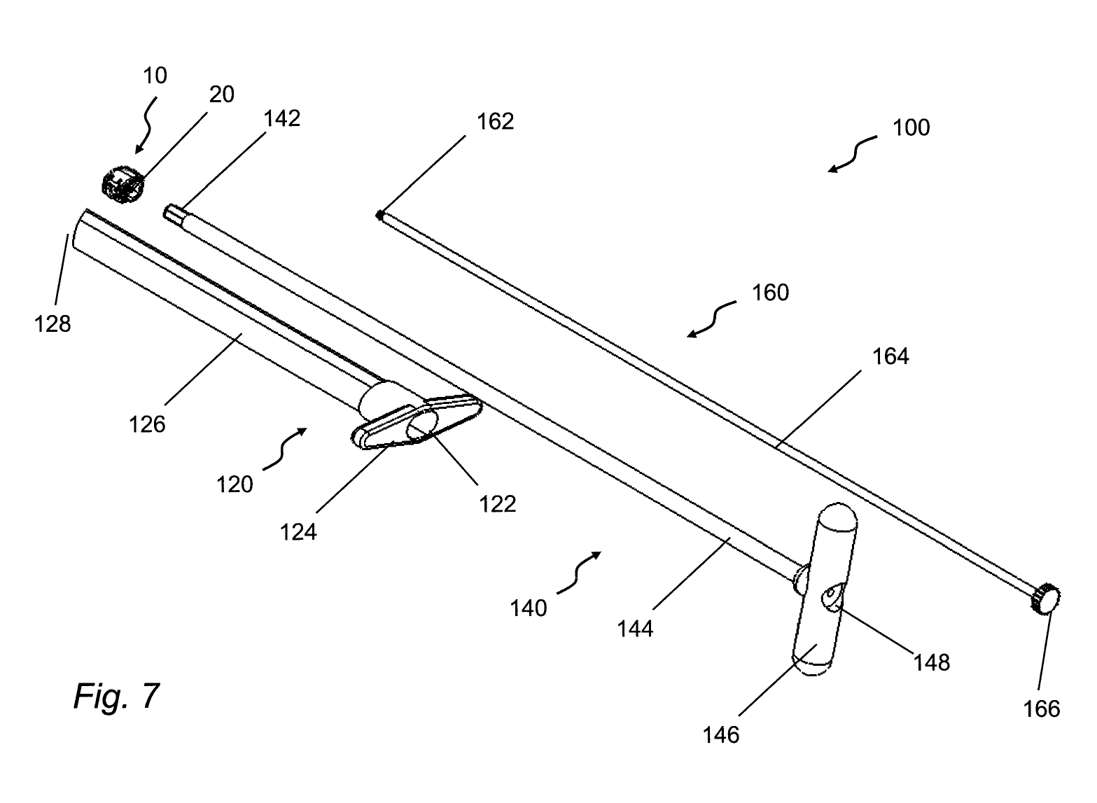

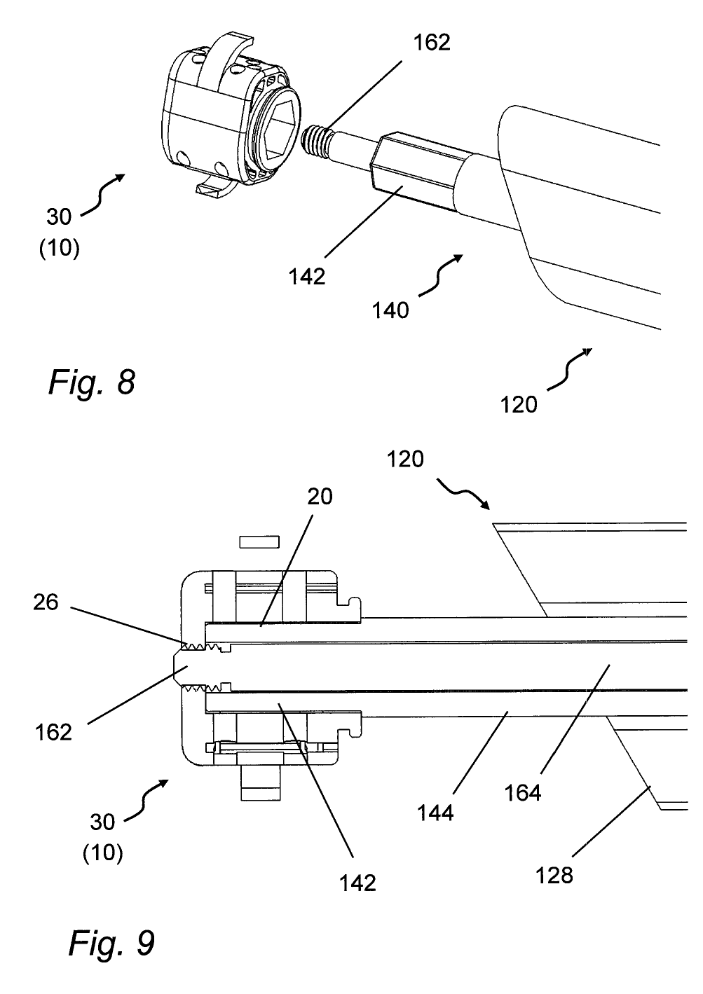

FIGS. 6, 7, 8, and 9 depict an delivery tool 100 for the rotating cam cages 10 that consists of a delivery sheath 120, a rotation handle 140, and a locking rod 160. The delivery sheath 120 has a hollow body 126 whose interior cross section 122 is shaped to allow passage of the rotating cam cage 10. The distal end 128 of the hollow body 126 may be angled such that an approximately equal amount of body will protrude through the disk wall (see FIG. 21). The proximal end of the hollow body 126 has a handle 124 to facilitate insertion and removal. The rotation handle 140 has a hollow shaft 144 that allows the locking rod 160 to pass completely through it. The distal end of the shaft 144 is formed to create an engagement feature 142 the fits into the corresponding structure 20 of the rotating cam cage 10 (shown as a typical hex shaft). The proximal end of the rotation handle 140 has a handle 146 that is used to rotate the rotating cam cage 10 into its final position after locating it within the disk space. The locking rod 160 is used to secure the rotating cam cage 10 to the rotating handle 140. It consists of a shaft 164 with a locking feature 162 (shown here as a threaded member) at its distal that engages corresponding features 26 in the rotating cam cage 10. A knurled knob 166 at the proximal end of the shaft 164 is used to release the rotating cam cage 10 from the rotating handle 140 once it has been properly placed in the disk space.

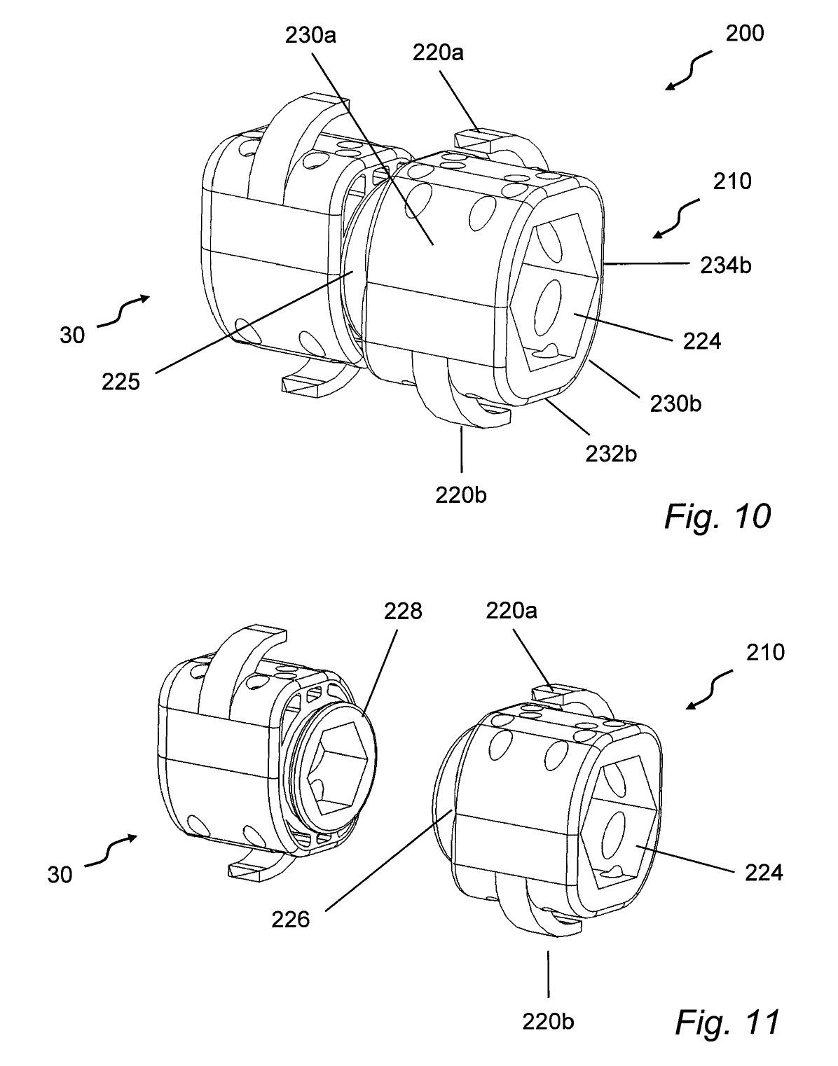

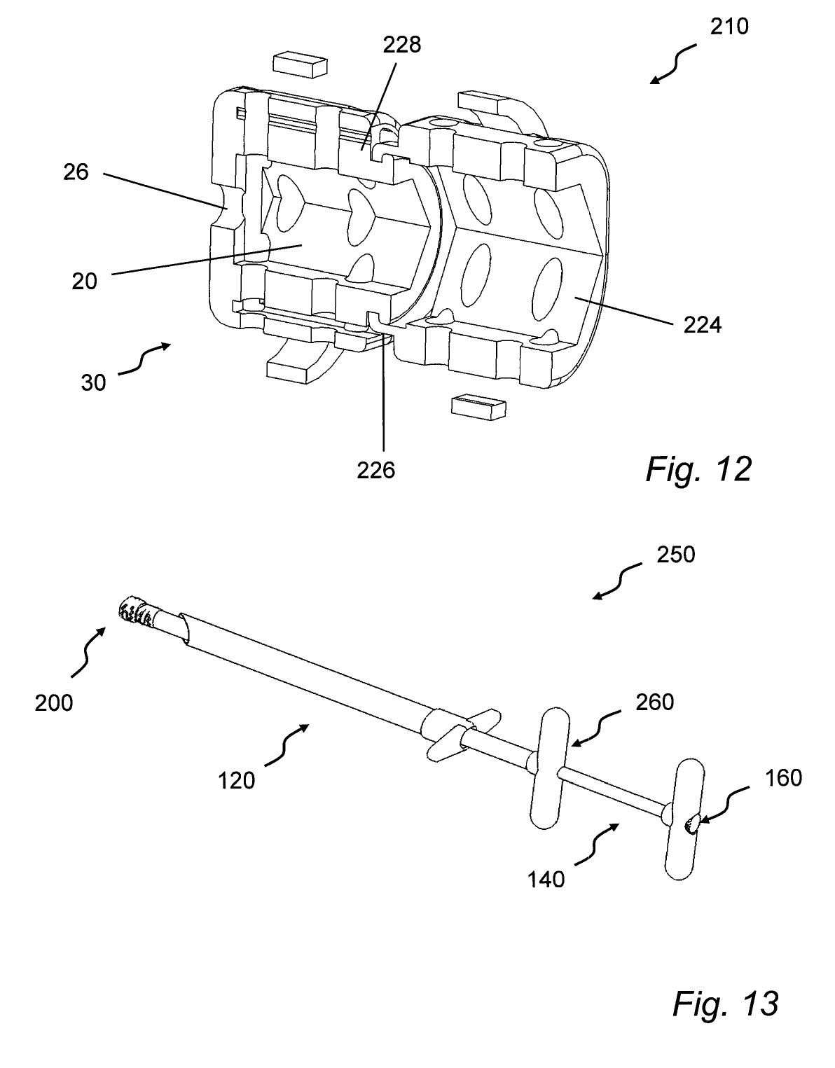

Depicted in FIGS. 10, 11, and 12, and herein defined as a counter-rotating cam cage 200 is an extension to the single rotating cam cages 10 and 30. Counter-rotating cam cage 200 combines the rotating cam cage 30 with an additional rotating cam cage 210 that is design to be rotated in the opposite direction for installation. The fixation anchors 220a and 220b face the opposite direction as their counterparts on rotating cam cage 30. Likewise, cam surfaces 230a and 230b are arranged to provide the cam/lifting action when the cam cage 210 is rotated in a counter-clockwise direction. The 2 counter rotating cam cages 30 and 210 are linked together through a rotation joint 225 that allows the cams to rotate relative to each other. The joint 225 can take various forms, here it is depicted as an undercut feature 228 on the cam 30 and a overlapping feature 226 on cam 210. Rotating cam cage 210 has an delivery tool engagement feature 224 that is similar to the one on cam 30 though increased in size. This allows it to engage with its rotational handle while at the same time allowing the rotational handle for the other cam 30 to engage it.

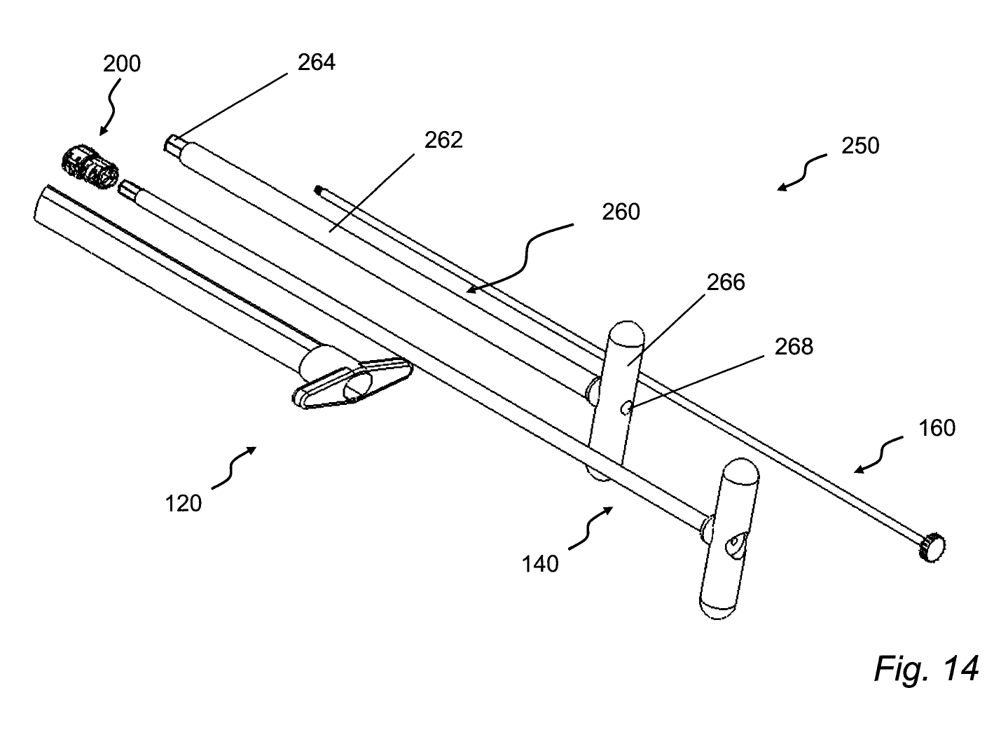

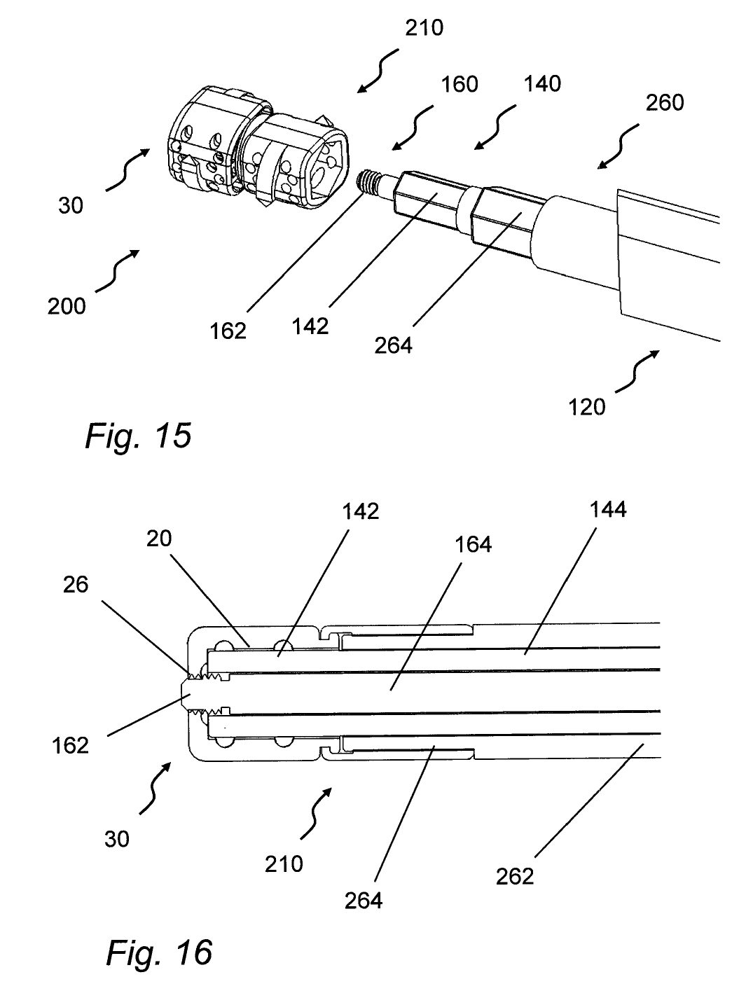

FIGS. 13, 14, 15, and 16 show the counter-rotating cam 200 assembled to its delivery tool 250. Delivery tool 250 is the same as delivery tool 100 with the addition of a second rotating handle 260 that engages with rotating cam cage 210. Rotating handle 260 consists of a hollow shaft 262 whose interior 268 is designed to fit over the shaft 144 of rotating handle 140. The distal end of shaft 264 is shaped to fit into the opening 224 of rotating cam cage 210. A handle 266 is affixed to the proximal end of shaft 262.

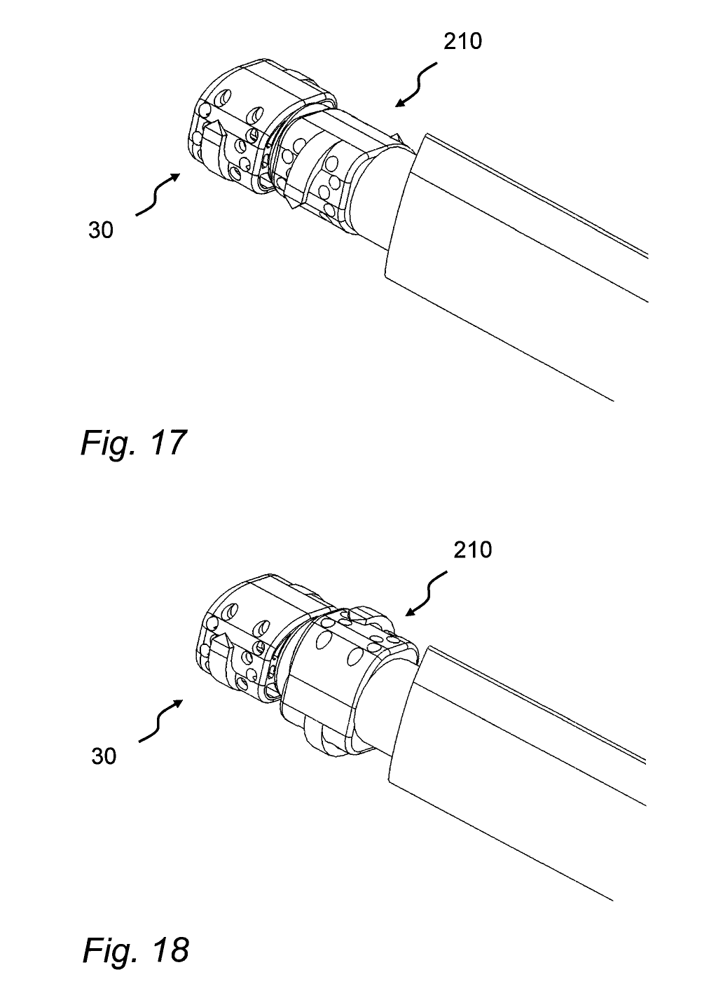

FIG. 17 depicts the counter-rotating cam cage 200, attached to its delivery tool 250, as it is first inserted into the disk space. FIG. 18 shows rotating cam cage 210 after it has been rotated 90 degrees counter clockwise while holding rotating cam cage 30 stationary, After rotating cam cage 210 is in position, held be the fixating anchors 220a and 220b, rotating cam cage 30 is rotated 90 degrees clockwise into its final position.

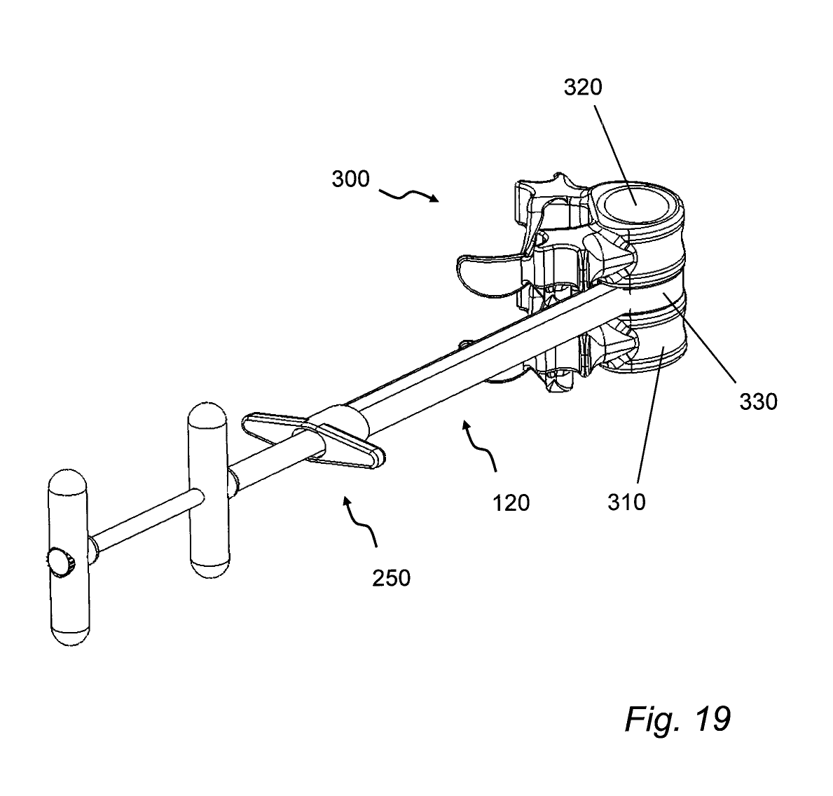

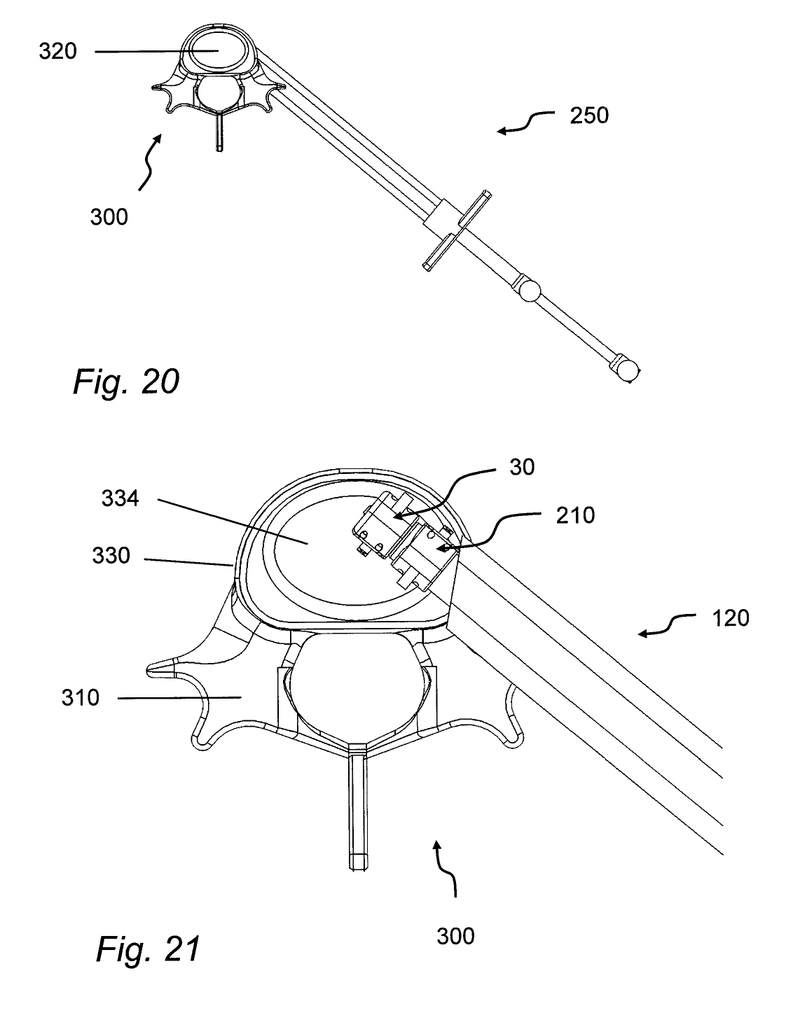

FIGS. 19, 20, and 21 illustrate the interaction of the delivery tool assembly 250 with a portion of the spine 300. The delivery sheath 120 passes through the outer tissue of the patients body and penetrates the side wall of the intended disk 330 which separates the upper disk 320 from the lower disk 310, Once the delivery sheath 120 is in place and the site preparation performed, the single rotating cam 10/30 or the counter-rotating cam cage 200 is passed through the delivery sheath 120 into the interior portion of the disk 334 where it is rotated into its final position. Once properly installed, the locking rod 160 disengages from the cam cage and is withdrawn along with the rotating handle(s).

The delivery tools 100 and 250 use manual force to rotate the rotating cam cages into position. An alternate embodiment would be to use a powered device to generate the rotational force. In particular a powered device that imparts rapid, measured rotational impacts (i.e. impact wrench), would provided for a controlled installation with less trauma to the boney plates of the vertebrae.

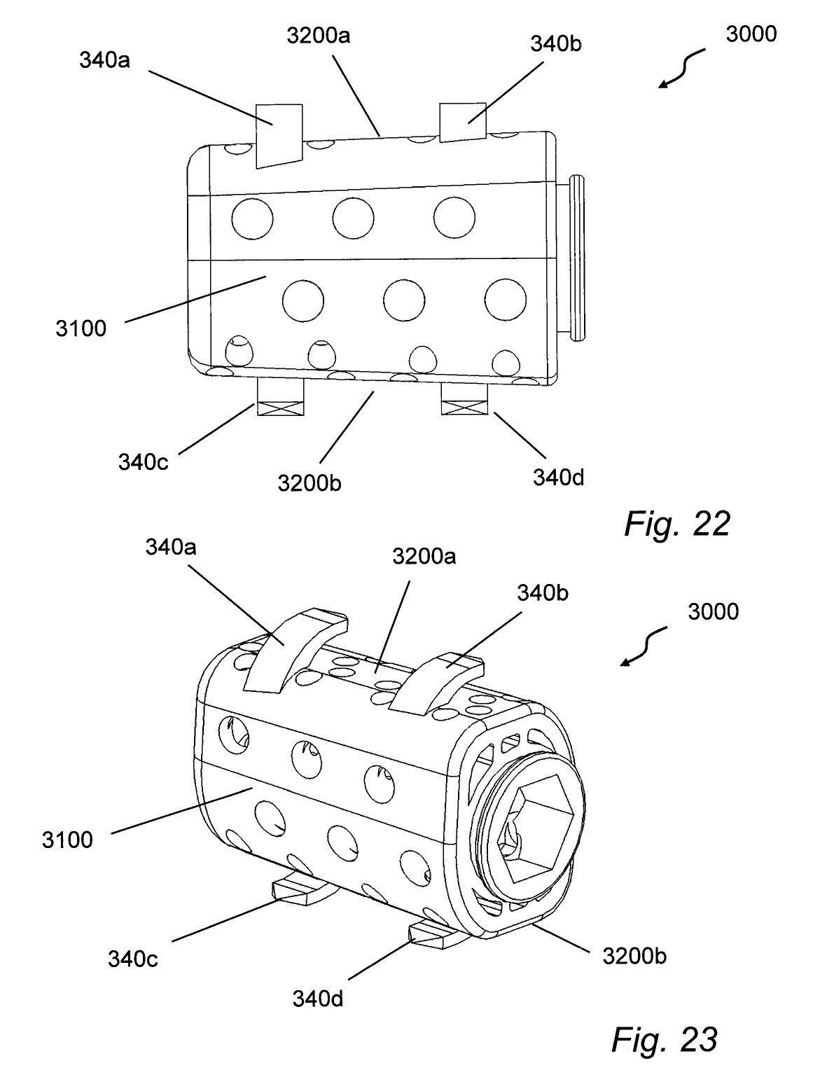

FIGS. 22 and 23 illustrate an alternate embodiment of the rotating cam cage designated 3000. This version shows the potential for 2 or more sets of fixation anchors 340a, 340b, 340c, and 340d. In addition, the cam body 3100 can have a different sized or shaped profile as it progresses from the distal to the proximal end. The cam body 3100 here tapers along the expanded planar surfaces 3200a and 3200b. The taper allows for more height increase at the proximal end.

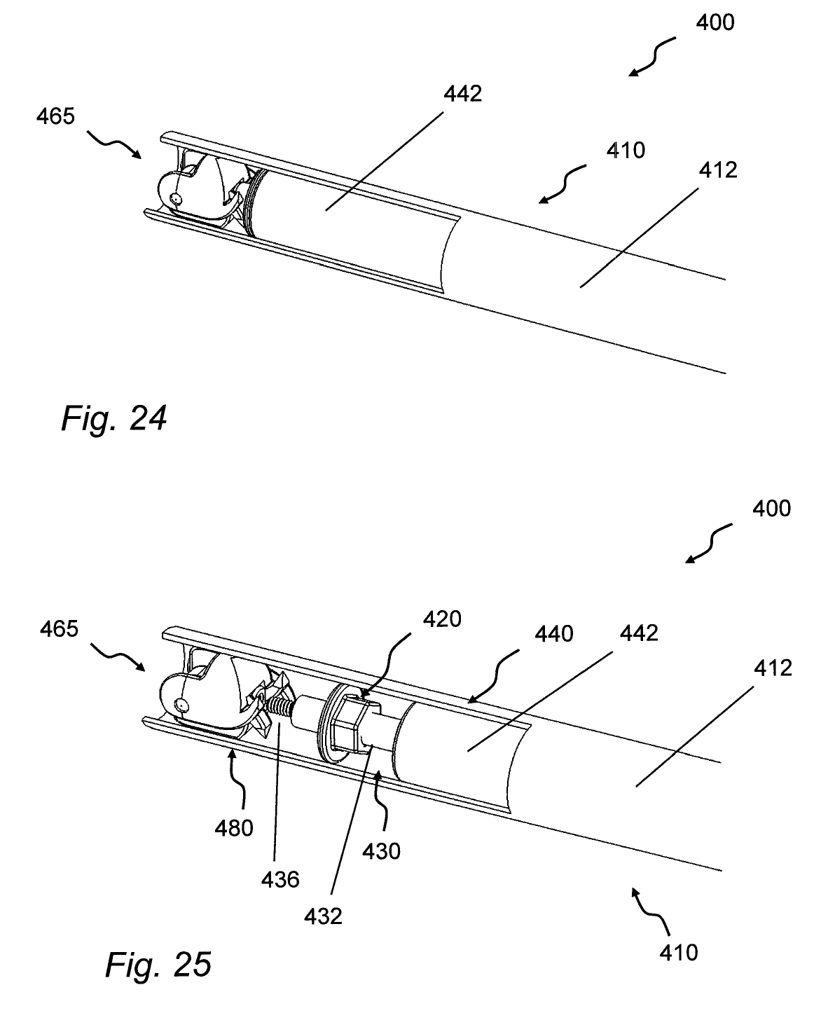

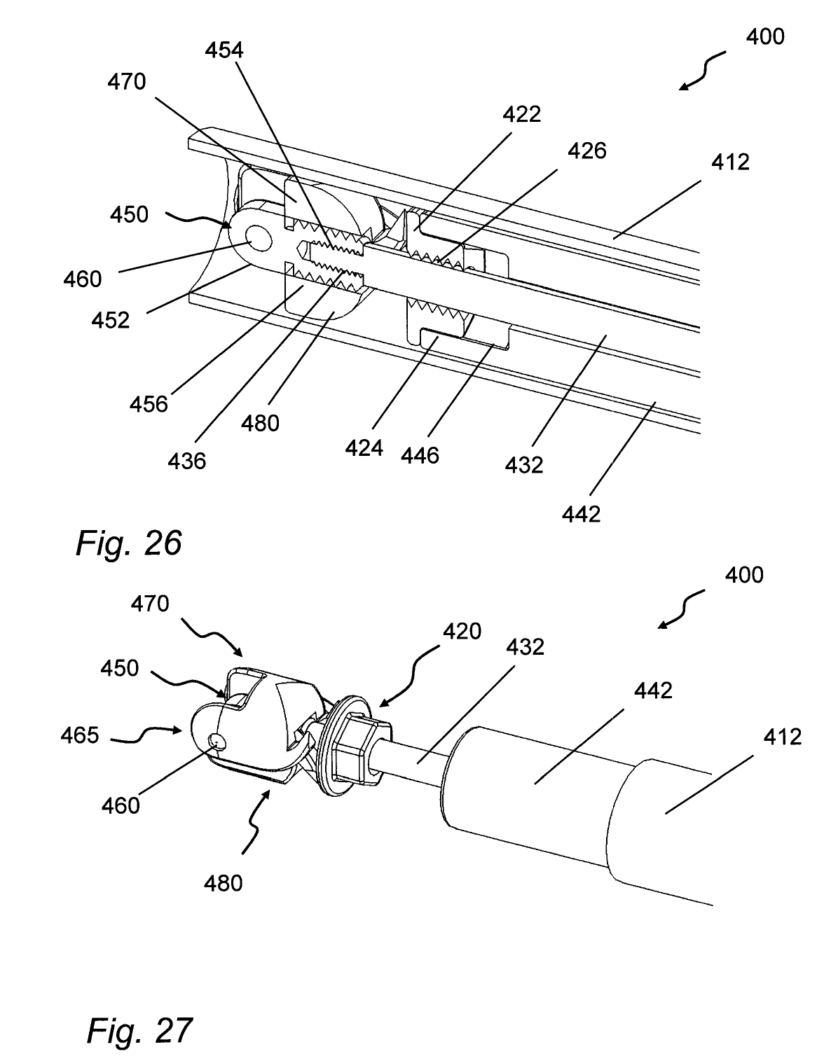

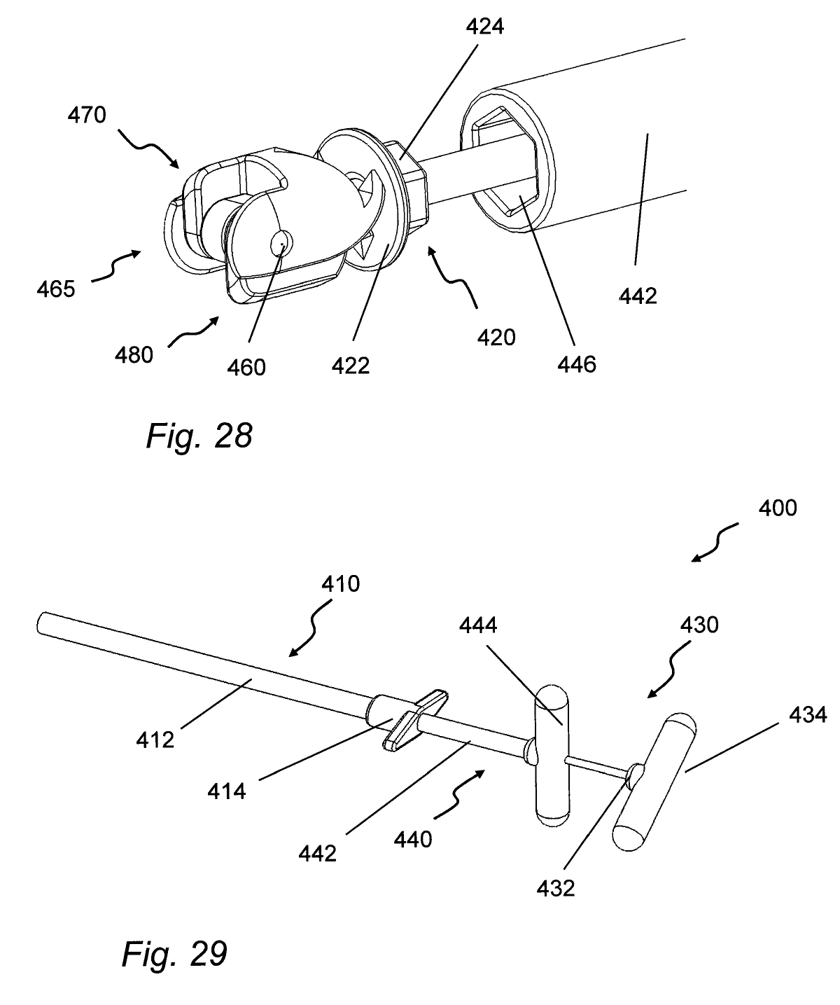

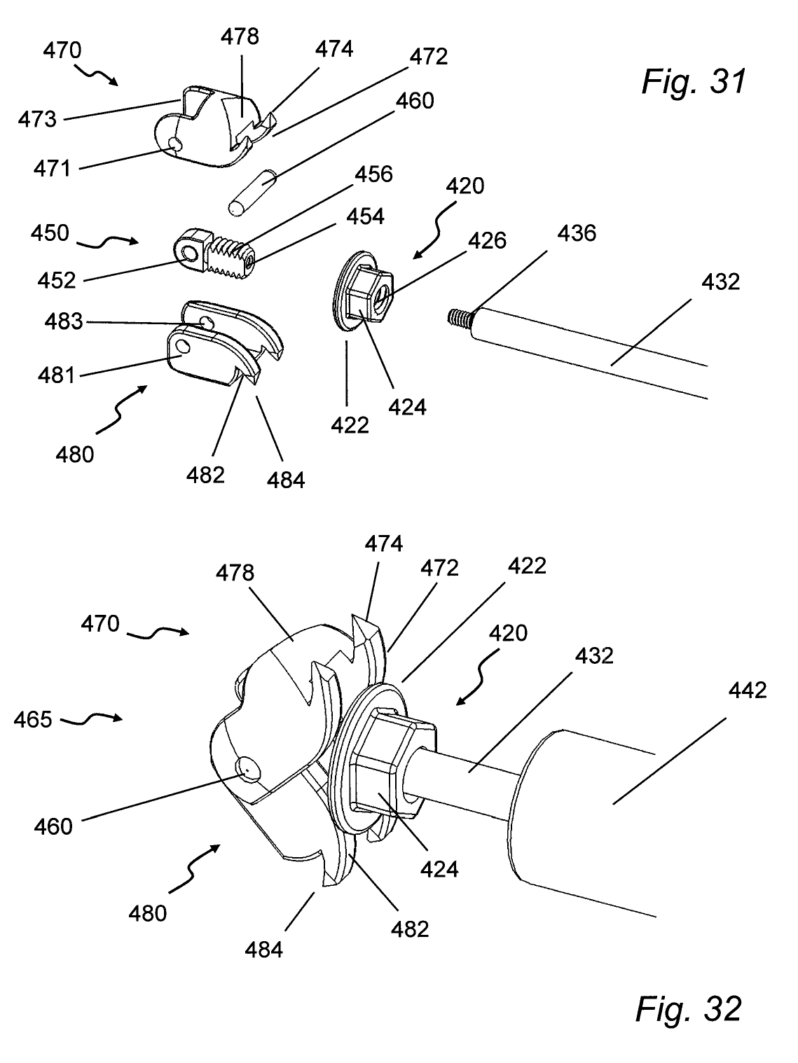

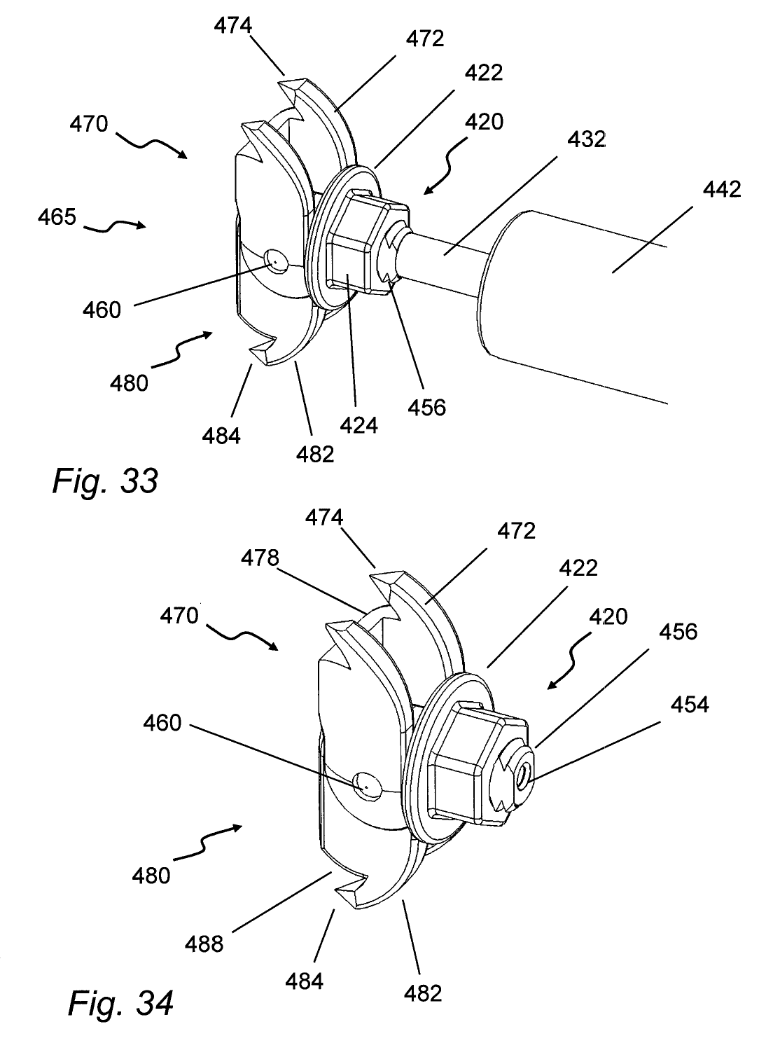

Referring to FIGS. 24 through 37, there is depicted an expanding cam assembly 465 with delivery sheath 410, installation rod 430, and nut driver 440 generally designated 400, fashioned in accordance with the present principles. FIG. 24 shows the expanding cam assembly 465 positioned inside the delivery sheath 410 as it would be during insertion into the disk space through the side wall of the disk. In FIG. 25, the nut 420, nut driver 440, and installation rod 430 have been exploded within the sheath 410 to illustrate their interaction. FIG. 27 depicts the expanding cam assembly 465 positioned outside of the delivery sheath 410 during the initial stage of the installation.

The expanding cam assembly 465 consists of 2 expanding cams 470 and 480, an anchor rod 450, a pivot pin 460, and a locking nut 420. The 2 expanding cam 470 and 480 shown in this embodiment are identical (rotated 180 degrees relative to each other as assembled). The expanding cam 470 and 480 has several defining features; a cam surface 478 and 488, fixation anchors 472 and 482, a slot 473 and 483, and a pivot pin hole 471 and 481. The pivot pin 460 captures each expanding cam 470 and 480 onto the anchor rod 450 as it passes through the expanding cam pivot pin holes 471 and 481 and the mating hole 452 in the anchor rod 450. The expanding cams 470 and 480 can pivot freely about the pivot pin 460. Additional features on the anchor rod 450 include external threads 456 that mate with the internal threads 426 of the locking nut 420 and internal threads 454 that mate with the external threads 436 of the installation rod 430. The final piece of the expanding cam assembly 465 is the locking nut 420 which consists of the aforementioned internal threads 426, an integral washer 422, and interfaces surfaces 424 that mate with corresponding surfaces 446 on the nut driver 440.

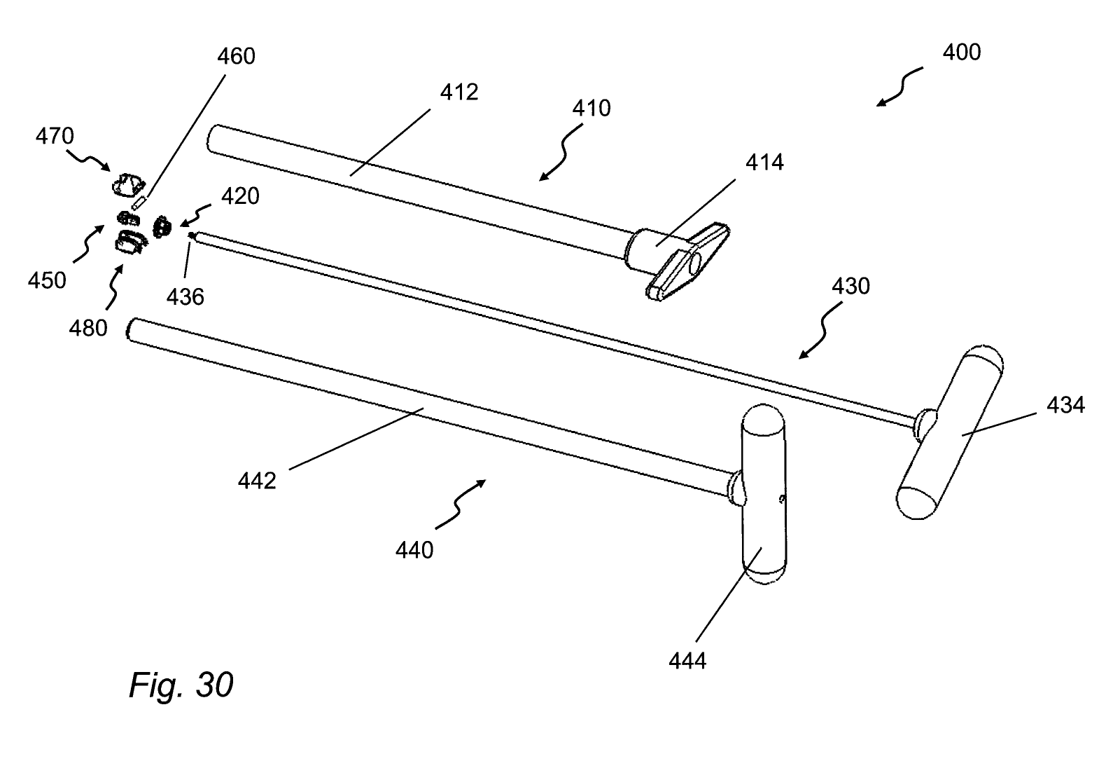

Referring to FIGS. 29 and 30, the delivery tool for the expanding cam assembly includes a delivery sheath 410, an installation rod 430, and a nut driver 440. The delivery sheath 410 consists of a hollow tube 412 sized to contain the expanding cam assembly 465 with an over-molded handle 414 for easily handling during insertion and removal. The next piece of the delivery tool assembly is the nut driver 440. Its hollow cylindrical body 442 fits within the sheath hollow tube 412. The distal end of the body 442 has internal surfaces 446 formed to mate with the external surfaces 424 of the locking nut 420 whereas, the proximal end contains a handle 444. The handle 444 is used to apply torque to the nut driver 440 which then transfers that torque to the locking nut 420 through the contact surfaces 424 and 446. This torque rotates the locking nut 420 which then translates over the threaded portion 456 of the anchor rod 450. The final piece of the delivery tool is the installation rod 430 which consists of a solid shaft 432 with a handle 434 on the proximal end and a threaded portion 436 on the distal end. The threaded portion 436 mates with the internal threads 454 of the anchor rod 450. The installation rod 430 holds onto the expanding cam assembly 465 during installation and then releases it by rotating the handle 434 of the installation rod 430 counter clockwise to unthread the distal end from the anchor rod 450.

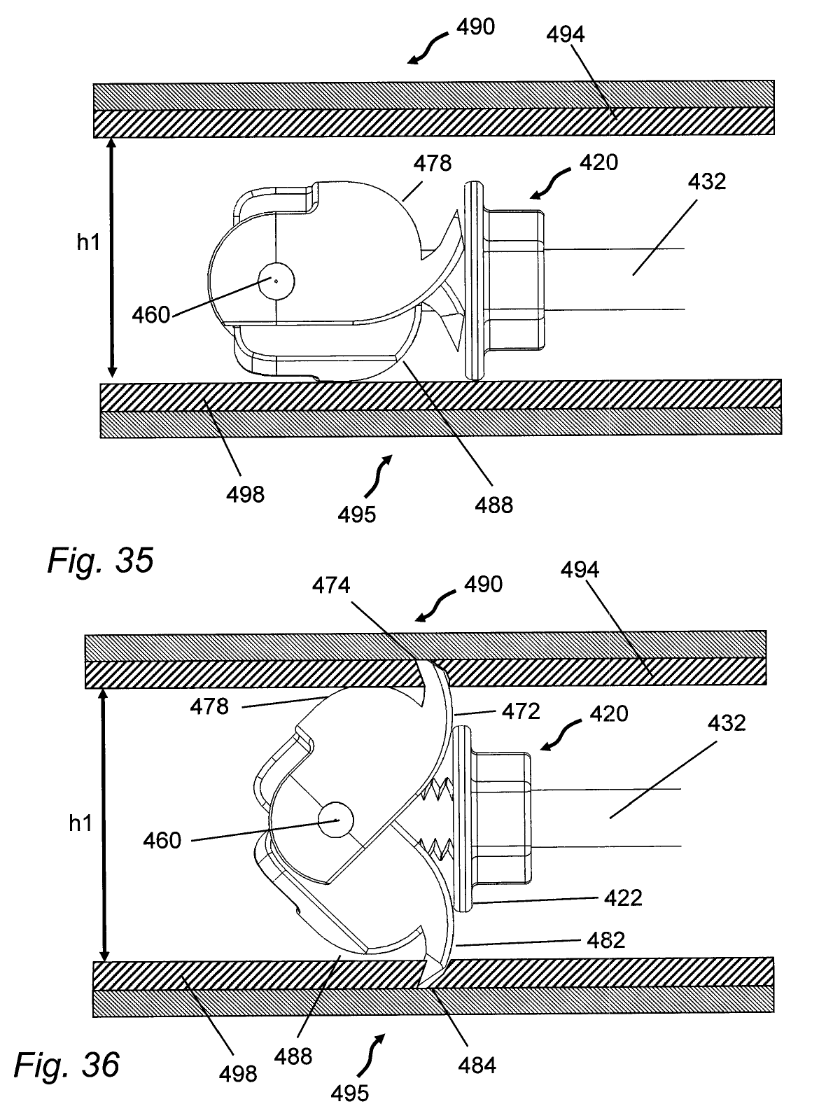

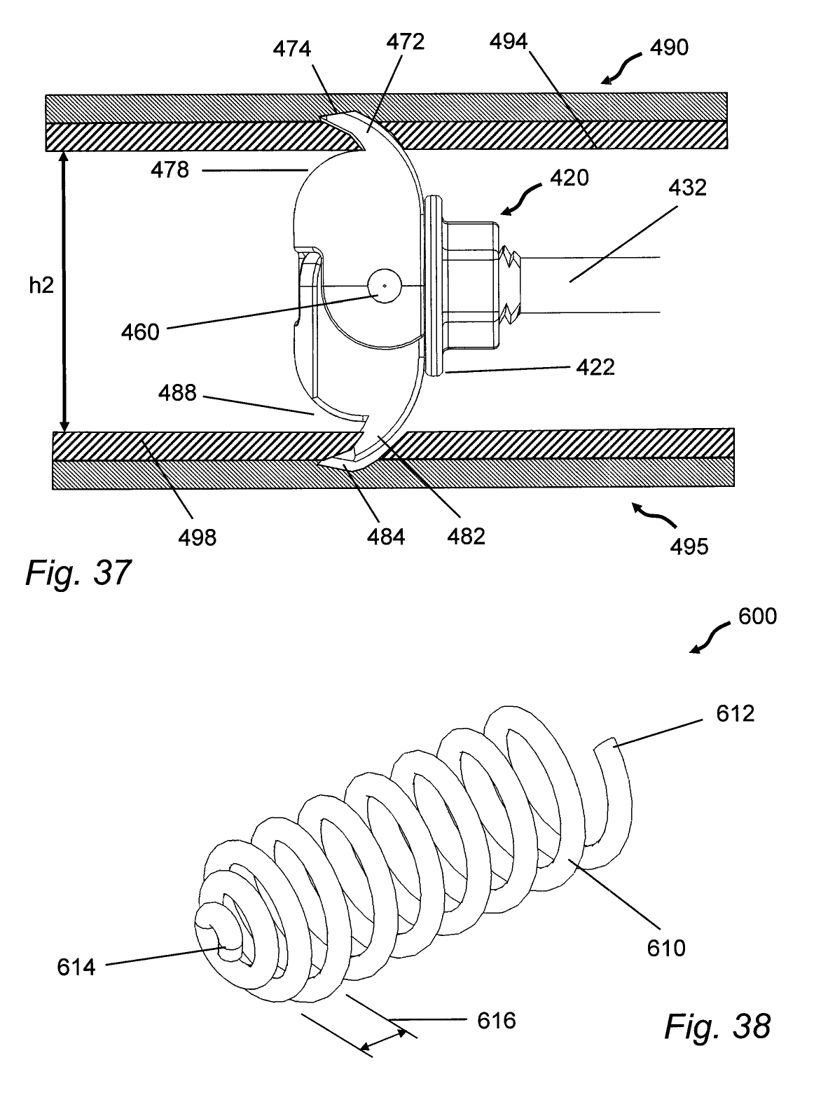

The expanding cam assembly 465 is installed within the disk space between 2 vertebrae by means of the delivery tool as follows: The complete assembly, expanding cam assembly 465 and delivery tool, are assembled as shown in FIGS. 24 and 29. Through an appropriate incision, the distal end assembly is inserted into the patient until the distal end of the delivery sheath 410 penetrates through the wall of the disk. The expanding cam assembly 465 is then extended out of the delivery sheath 410 as shown in FIG. 27 until position at the desired location in the disk space. Torque is applied to the handle 444 of the nut driver 440 while holding the handle 434 of the installation rod 430 stationary. Rotating the handle 444 of the nut driver 440 will cause the locking nut 420 to rotate relative to the anchor rod 450 thus translating the locking nut 420 over the anchor rod 450 due to the mating threads 426 and 456. As the locking nut 420 translates, the integral washer 422 will contact the curved surface of the fixation anchors 472 and 482 of the cams 470 and 480 forcing the cams 470 and 480 to rotate in opposite directions about the pivot pin 460 (see FIG. 32). The cams 470 and 480 will continue to rotate unimpeded until the sharp tips 474 and 484 of the fixation anchors 472 and 482 or the cam surfaces 478 and 488 contact the upper and lower plates 494 and 498 of the 2 adjacent vertebral bodies 490 and 495 (see FIGS. 35 through 37). As additional torque is applied to the nut driver 440, the locking nut 420 forces the expanding cams 470 and 480 to continue to rotate. This additional rotation applied a separating on the 2 vertebral bodies 490 and 495 through the interaction of the cam surfaces 478 and 488 on the vertebral plates 494 and 498. The shape of the cam surfaces 478 and 488 is such that it provides a smooth, gentle force. The initial separation of the vertebral bodies shown as distance “h1” in FIGS. 35 and 36 is increased to “h2” shown in FIG. 37 as the expanding cams 470 and 480 reach their final position. In addition to the separation force caused by the cam surfaces 478 and 488, a piercing force delivered at the sharp ends 474 and 484 of the fixation anchors 472 and 482 causes the fixation anchors 472 and 482 to penetrate the plates 494 and 498 of the vertebral bodies 490 and 495 as the rotation occurs. When the expanding cams 470 and 480 reach their final positions, the fixation anchors 472 and 482 will have been embedded within the plates 494 and 498 creating a mechanical fixation between the 2 vertebral bodies 490 and 495. Once the locking nut 420 forces the expanding cams 470 and 480 into their final position the installation rod 430 is rotated to unthread itself from the anchor rod 450 allowing the delivery tool (installation rod 430 and nut driver 440) to be removed proximally through the delivery sheath 410. At this point, the delivery sheath 410 can be removed or left in place to allow another expanding cam assembly 465 to be placed through it.

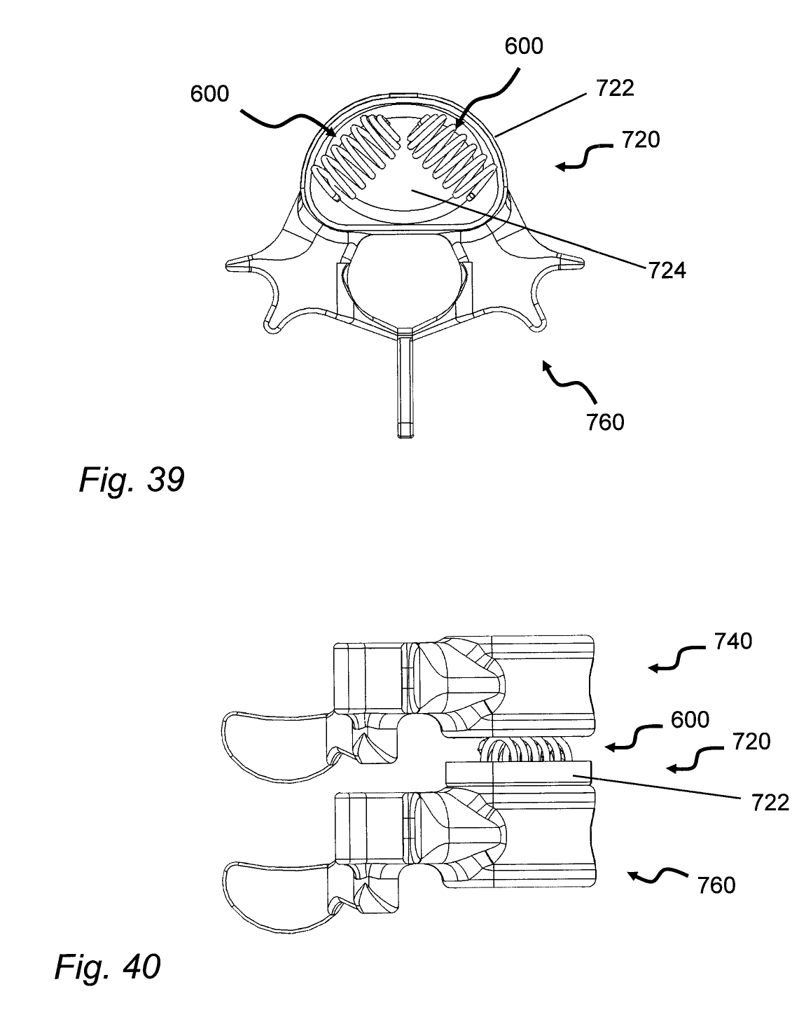

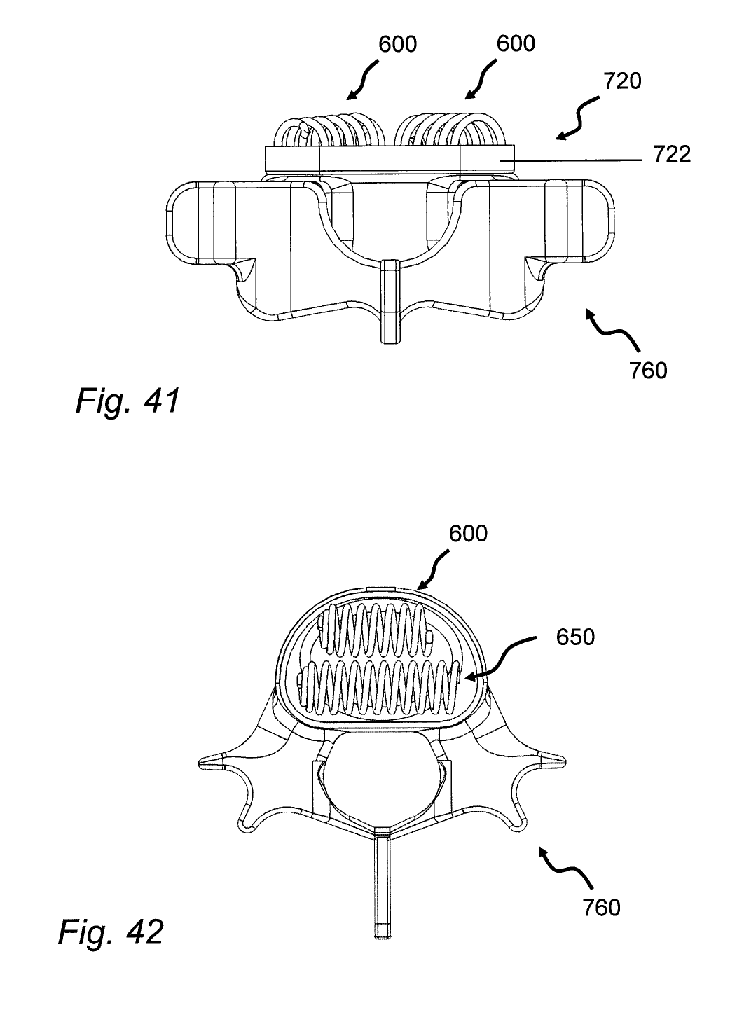

Referring to FIG. 38, there is depicted a spring cage generally designated 600, fashioned in accordance with the present principles. The spring cage consists of a single structure, spring body 610 which may be formed in various manners from an appropriate bio-compatible material such as stainless steel, nitinol, or a polymeric material. The body of the spring cage 600 is formed from a single wire in a helical form with a defined outside diameter, wire cross section diameter, pitch length 616 (coil to coil spacing), and number of turns. The distal end 614 of the spring cage 600 may be formed in a closed manner to create a tapered end. The proximal end 612 may end abruptly as shown or may have a formed turn-in to eliminate a sharp edge. FIGS. 39-41 show 2 of the spring cages 600 deployed within the disk 720 between 2 adjacent vertebrae 740 and 760. They are inserted into the disk space 724 of the disk 720 through the side wall 722. The outside diameter of the spring body 610 is defined such that it is larger than the separation between the adjacent vertebrae 740 and 760 so that the spring cage 600 applies a separation force to correct any compression of the disk that may have occurred.

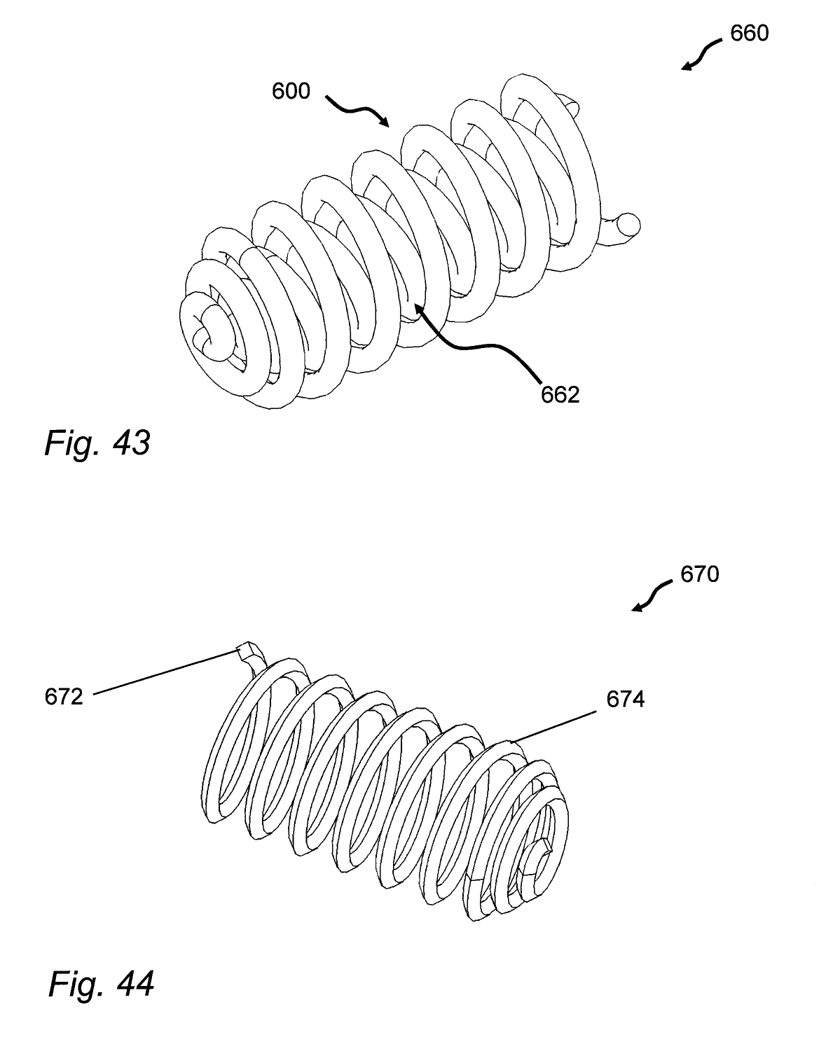

FIG. 42 shows an alternate arrangement wherein one spring cage 600 is installed with an elongated version of the spring cage 650 in a parallel fashion.

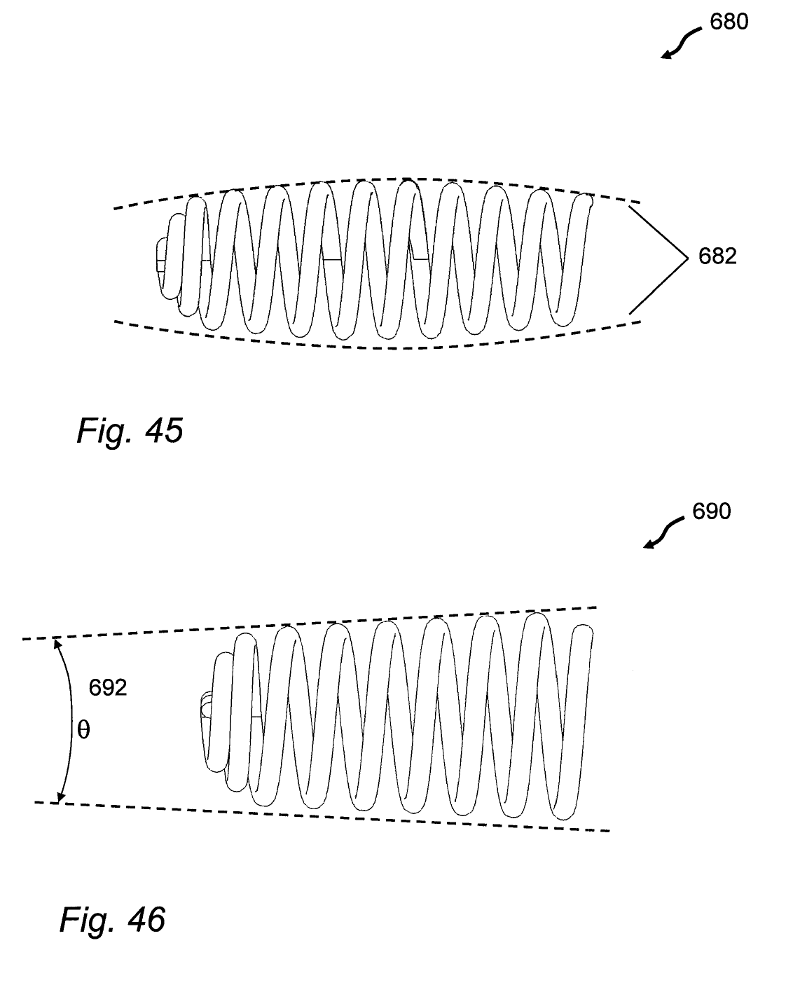

FIG. 43 shows an alternated embodiment of the spring cage 660 where a second spring cage 662 has been deployed within the first spring cage 600. The second spring cage 662 would have an outside diameter somewhat larger the inside diameter of the first spring cage 600 providing structural support to it. Additional spring cages could be placed within this assembly if desired. The second spring cage 662 could have an opposite hand (counter-clockwise versus clockwise) for the helical shape or the same hand. Having an opposite hand would create a lattice type shell effect helping to contain any biologic material that may be inserted into the interior of the spring cages. It should be noted that the spring cages could have different materials, cross-section shapes, pitches, and number of turns as desired.

FIG. 44 shows an alternated embodiment of the spring cage 670 wherein the cross-section shape 672 is non-circular. In this example, the cross section 672 is square with an edge of the square position to the outside surface 674 creating screw thread type effect.

FIG. 45 shows an alternated embodiment of the spring cage 680 that has an external contour 682 that is non-cylindrical. It should be noted that the external envelope or shape can vary in size with each turn symmetrically or non symmetrically, as desired. This could be advantageous in forming to the contours of the non planar vertebral plates.

FIG. 46 shows an alternated embodiment of the spring cage 690 that has an external contour 692 that is tapered (larger in the proximal section). This could be advantageous in applying variable force to the vertebral plates.

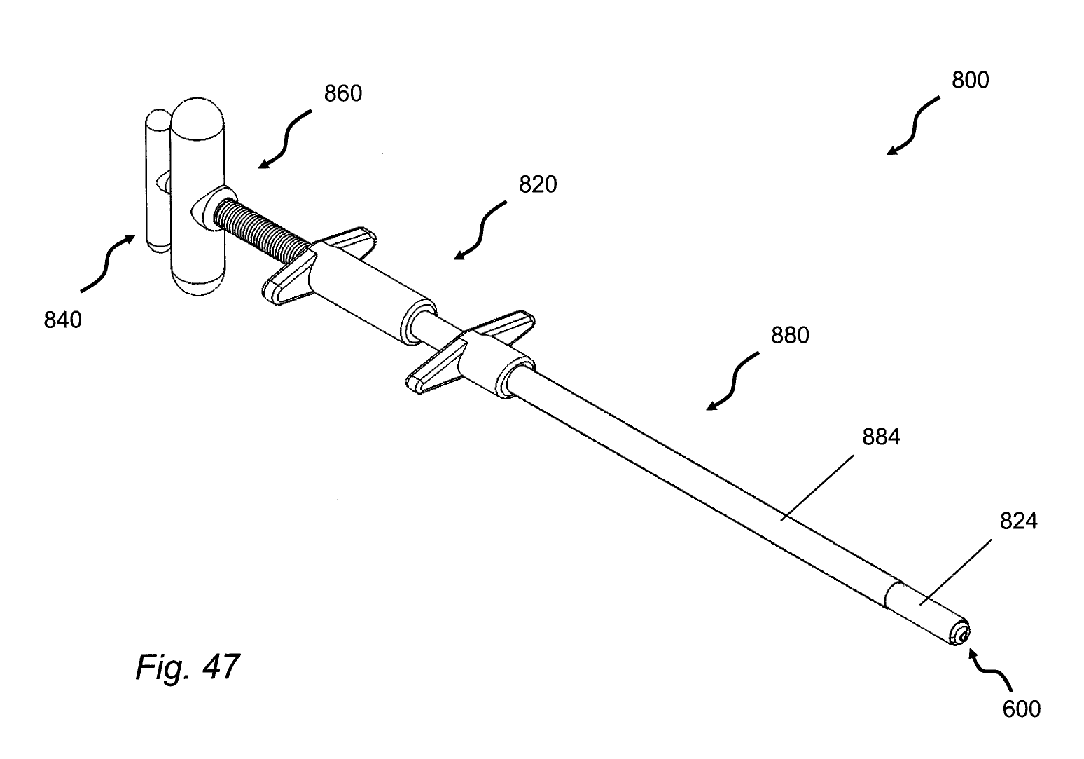

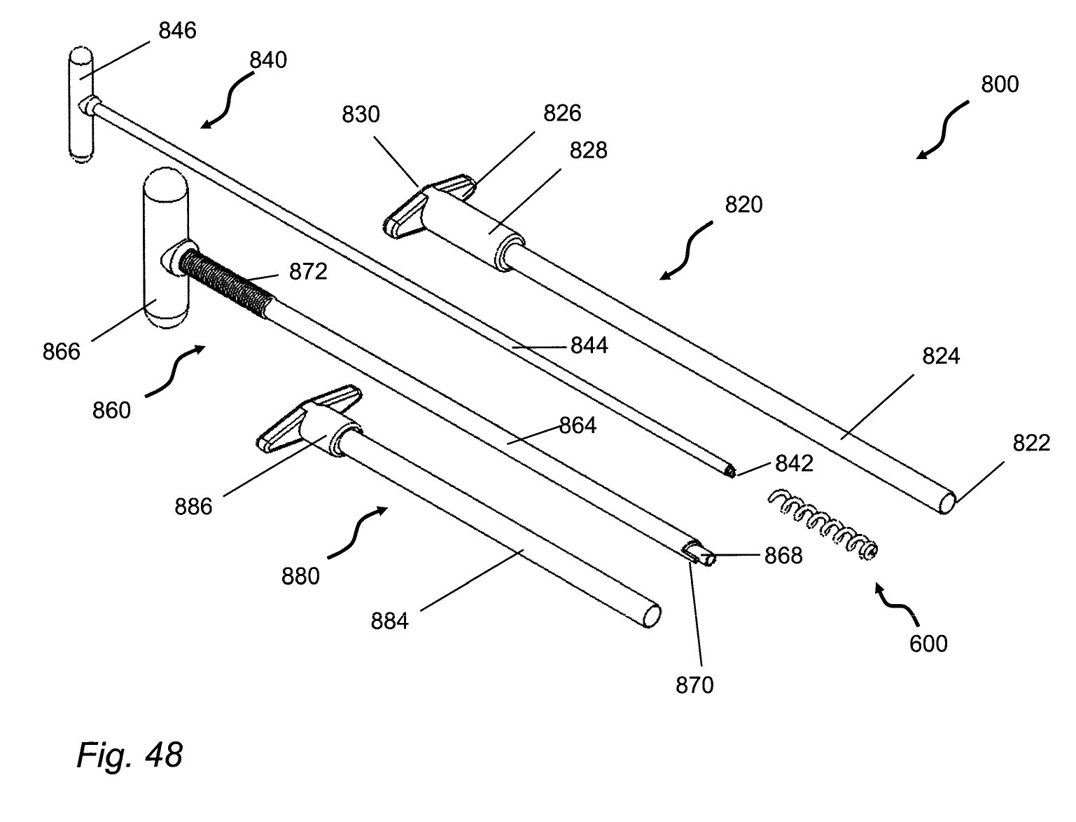

Referring to FIGS. 47 and 48, the delivery tool 800 for the spring cage 600 includes a delivery sheath 880, an introducer tube 820, a distal pusher deployment rod 840, and a proximal pusher deployment rod 860. The delivery sheath 880 consists of a hollow tube 884 with an over-molded handle 886 for easily handling during insertion and removal. The second piece of the delivery tool assembly is the introducer tube 820. Its hollow cylindrical body 824 fits within the sheath hollow tube 884. The diameter of the hollow interior 822 of the introducer tube 820 is smaller than the outside diameter of the spring cage 600. The spring cage 600 is squeezed radially and elongated axially to fit within this interior cylindrical space. The proximal end of the introducer tube 820 has a formed handle 826 with a cylindrical body 828 that contains internal threads 830. The internal threads 830 mate with the external threads 872 of the next piece of the delivery tool, the proximal pusher deployment rod 860. The proximal pusher 860 consists of a hollow shaft 864 with a handle 866 and external threads 872 at its proximal end. The distal end of the proximal pusher 860 contains a cylindrical section 868 that fits within the inner diameter of the compressed spring cage 600 and a drive wall 870 that mates with the proximal end 612 of the spring cage 600. The final component of the delivery tool 800 is the distal pusher deployment rod 840. It features a solid shaft 844 with a formed handle 846 at the proximal end and an interface structure 842 at the distal end. The interface structure 842 is formed to mate with the distal end geometry 614 of the spring cage 600.

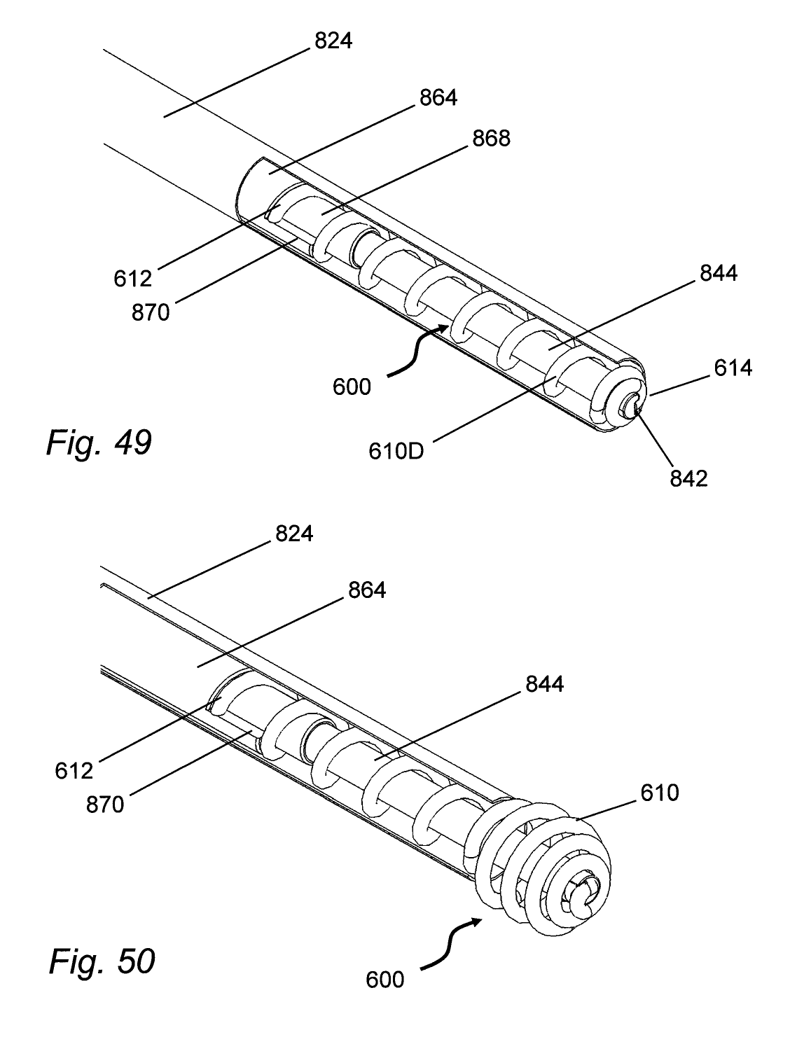

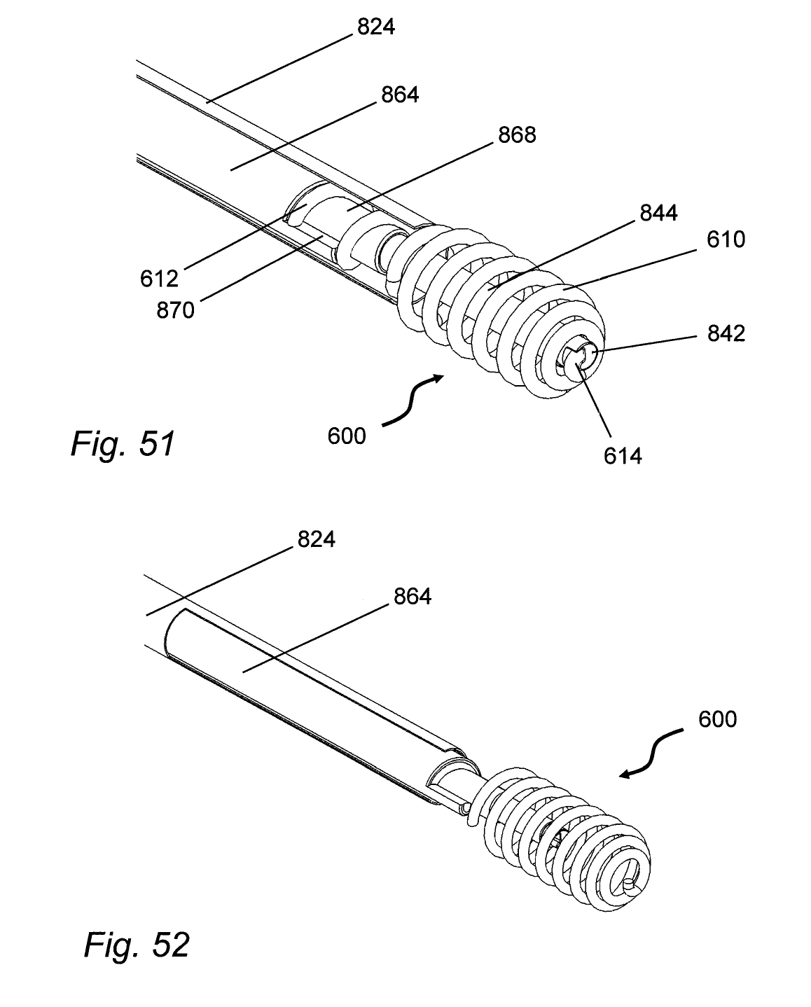

In use, the delivery sheath 880 is passed through the external tissue of the body and through a sized opening in the disk wall where it acts as a conduit for the rest of the delivery tool. The introducer tube 820 with the spring cage 600, proximal pusher 860, and distal pusher 840 assembled within it is inserted through the delivery sheath 880 until the distal end of the introducer 820 is positioned at the desired location within the disk space. FIGS. 49, 50, 51, and 52 illustrate the deployment sequence for the spring cage 600 (a section of the introducer wall is removed for clarity). FIG. 49 shows the spring cage 600 in its pre-deployment state with compressed spring body 610D. To deploy, the proximal and distal pushers 860 and 840 are rotated relative the introducer tube 820. The mating threads 872 and 830 of the proximal pusher 860 and the introducer tube 820 drive the pushers 860 and 840 axially within the introducer tube 820. The axial translation of the pusher 860 and 840 drive the spring cage 600 out the end of the introducer tube body 824 allowing the spring cage body 610 to expand to its original diameter while within the disk space (see FIG. 50, 51, 52). In addition, the rotation of the pushers 860 and 840 relative to the introducer tube 820 caused the spring cage 600 to rotate relative to the introducer tube 820 as well. This rotation acts to help draw the coils of the spring cage 600 out the end of the introducer tube 820.

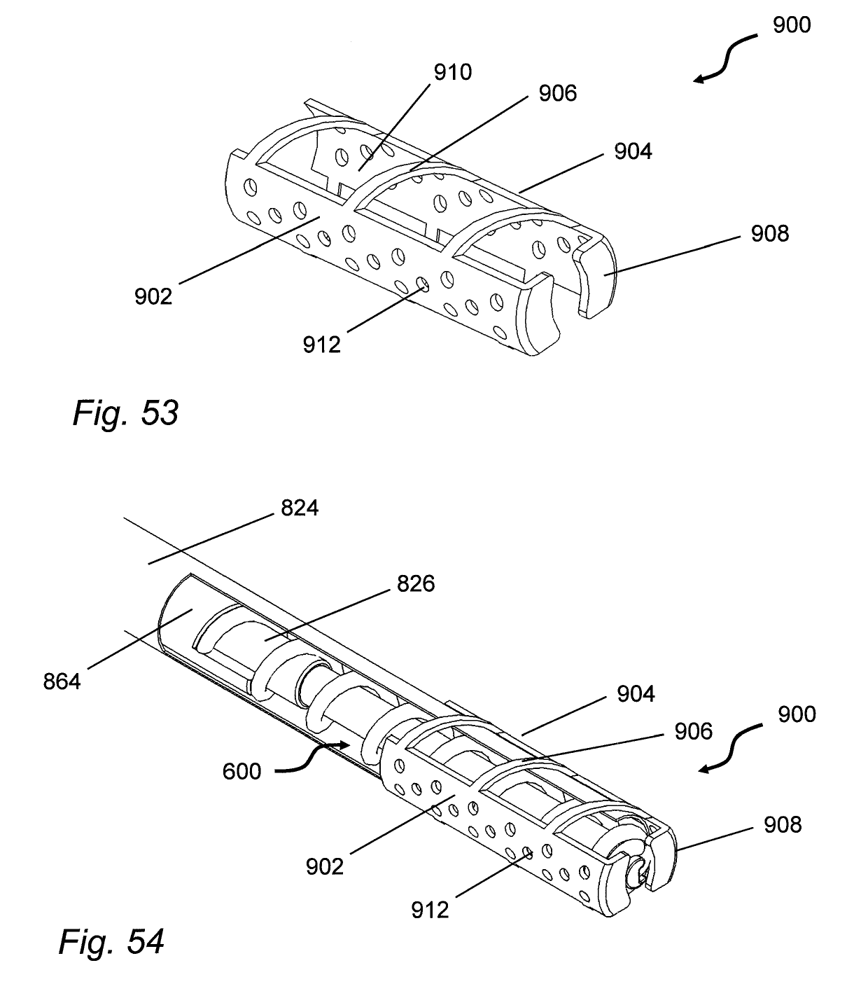

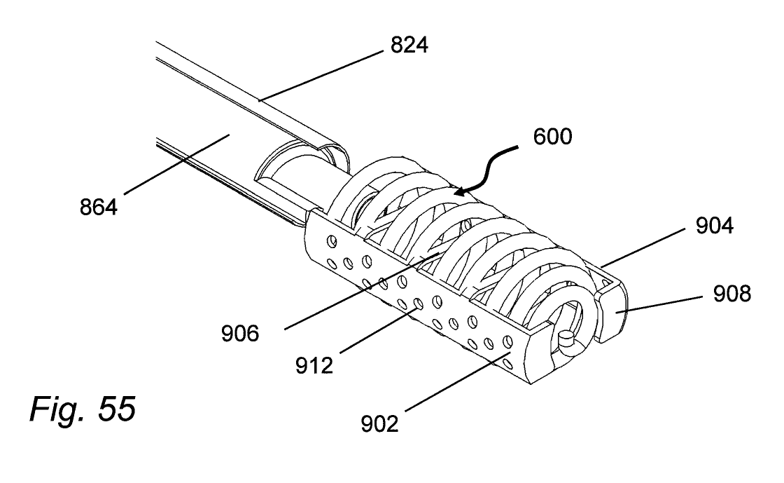

Referring to FIG. 53, there is depicted a containment cage generally designated 900 fashioned in accordance with the present principles. The containment cage consists of a single structure which may be formed in various manners from an appropriate bio-compatible material such as stainless steel, nitinol, or a polymeric material (e.g. PEEK polymer). The body of the containment cage 900 contains 2 side walls 902 and 904 that are connected with a number of bridging arms 906. Side perforations 912 penetrate both side walls 902 and 904. The exterior envelope of the side walls 902 and 904 and the bridging arms 906 is cylindrical in shape in its as-constructed shape. The distal ends of the side walls 902 and 904 have formed end plates 908. FIGS. 54 and 55 show the containment cage 900 in place over the distal end of the introducer tube 820 which contains the spring cage 600 (a section of the introducer tube is removed for clarity). The interior cylindrical shape of the containment cage matches the exterior shape of the introducer tube 820 such that it fits snuggly in place. As deployment of the spring cage 600 takes place (see FIGS. 49 through 52), the end plates 908 of the containment cage 900 contact the distal end of the spring cage 600 driving the containment cage 900 off of the end of the introducer tube 820 onto the spring cage 600. As the spring cage 600 expands to its original diameter, the side walls 902 and 904 expand with the spring cage body 610. the bridging arms 906 are deformed to a near flat shape to allow the side walls 902 and 904 to expand outward. Once fully deployed, the containment cage 900 acts as an integral sidewall containment for the spring cage 600 for biologic material that is placed inside the spring cage 600. The side walls prevent leakage of the biologic material through the sides of the spring cage into the disk space; however, the material can still make integral contact with the vertebral plates out the top and bottom of the spring cage. Side perforations 912 allow bone growth through and around the side wall 902 and 904.

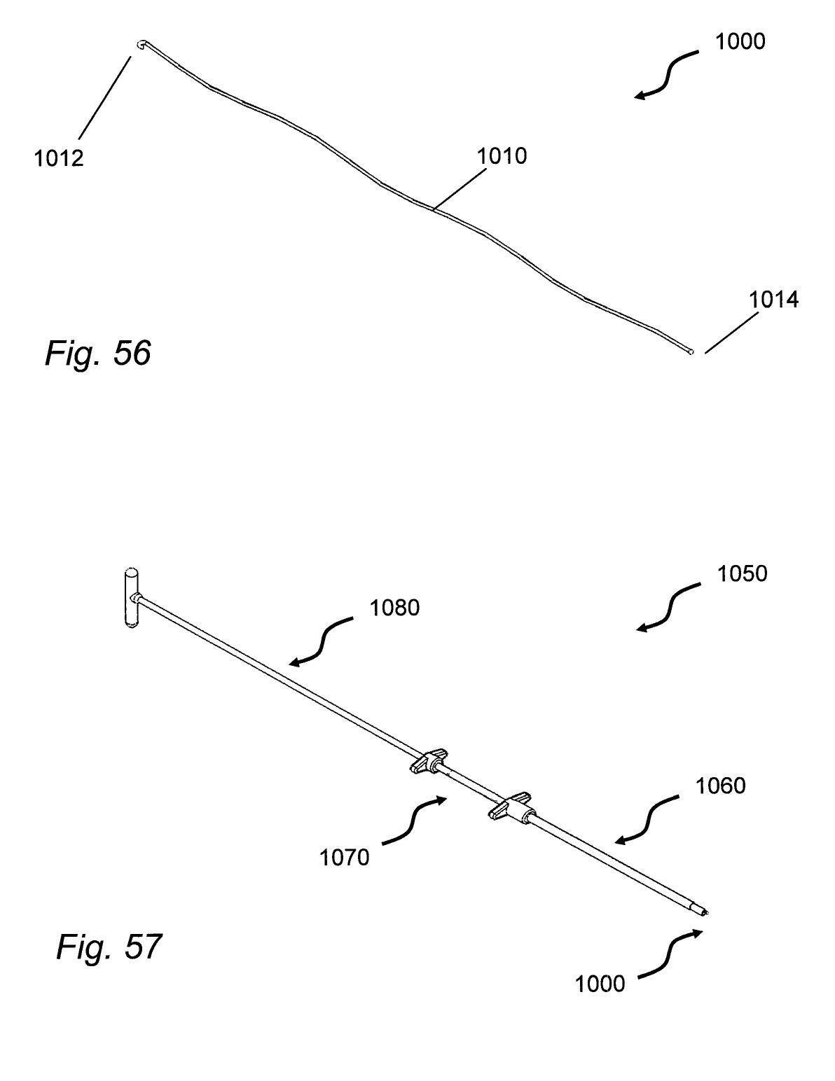

Referring to FIG. 56, there is depicted a random coil support device generally designated 1000, fashioned in accordance with the present principles. The random coil support device consists of a single structure, coil body 1010 which may be formed in various manners from an appropriate bio-compatible material such as stainless steel, nitinol, or a polymeric material (e.g. PEEK polymer). This embodiment of the random coil support device 1000 is formed from a single wire in a helical form with a defined outside diameter, wire cross section diameter, pitch length, and total length. The distal end 1014 of the random coil support device 1000 may be formed, or have a secondary part affixed to it, to create a blunted end. The proximal end 1012 is formed to create shape facilitating the delivery of the device.

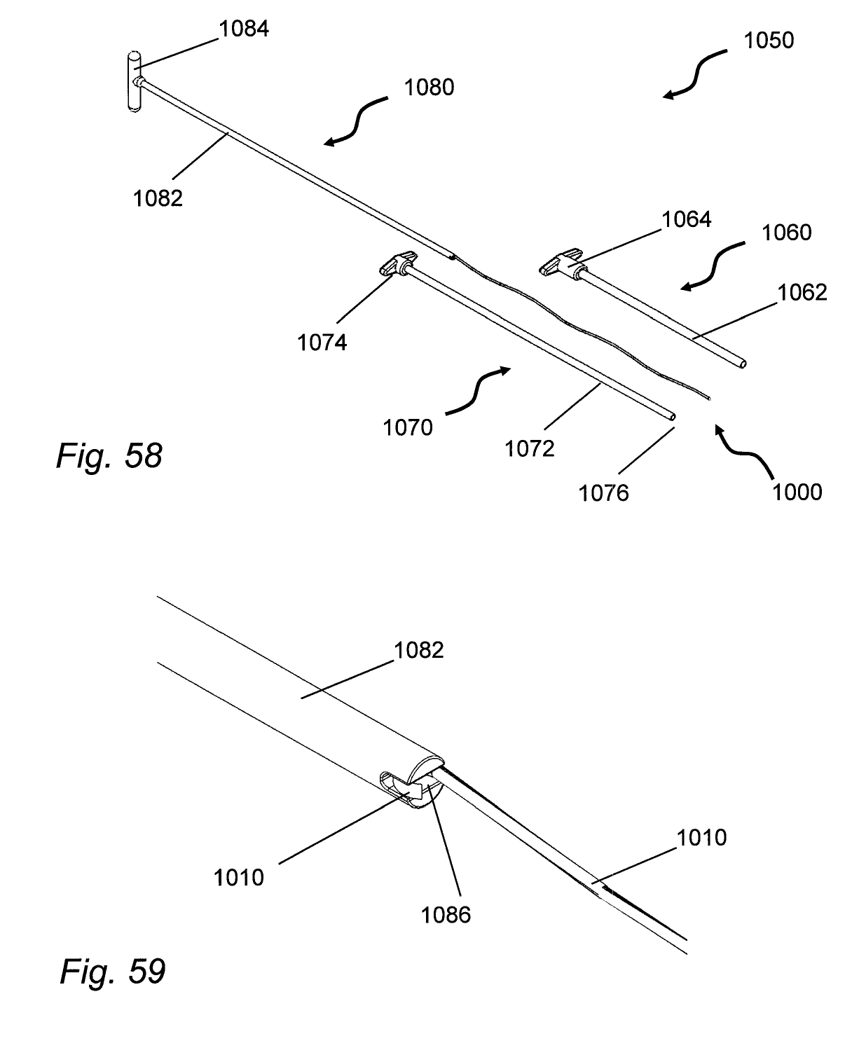

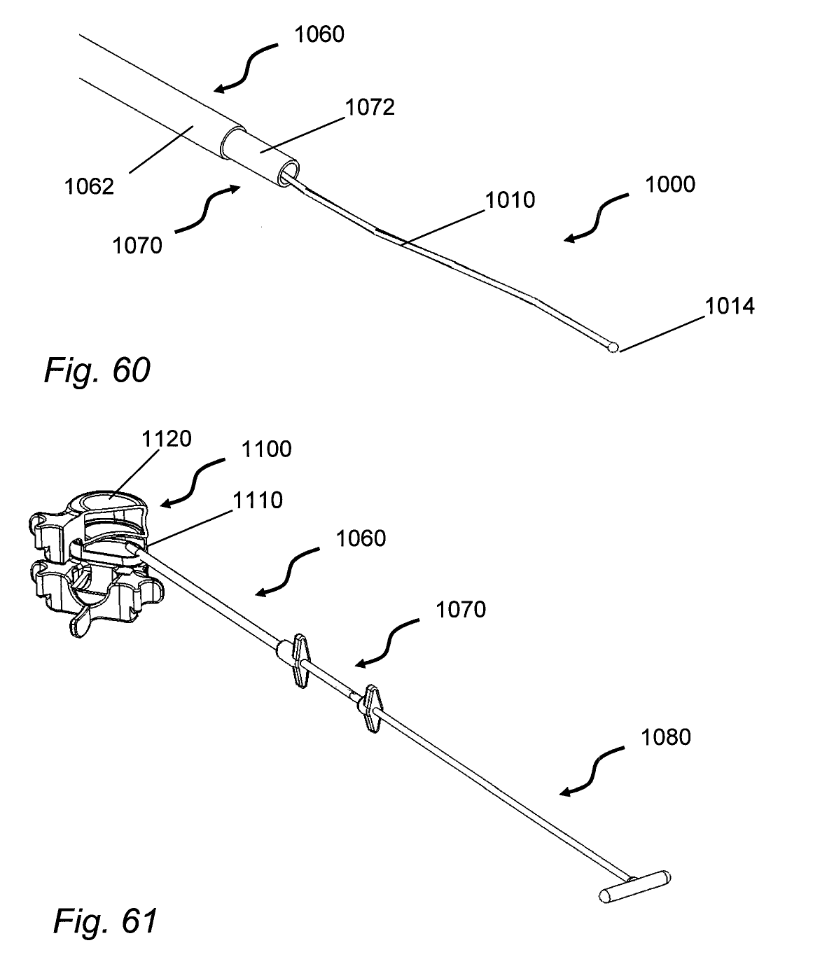

Referring to FIGS. 57, 58, 59, and 60, the delivery tool 1050 for the random coil support device 1000 includes a delivery sheath 1060, an introducer tube 1070, and a deployment rod 1080. The delivery sheath 1060 consists of a hollow tube 1062 with an over-molded handle 1064 for easily handling during insertion and removal. The second piece of the delivery tool assembly is the introducer tube 1070. Its hollow cylindrical body 1072 fits within the sheath hollow tube 1062. The proximal end of the introducer tube 1070 has a formed handle 1074. The final component of the delivery tool 1050 is the deployment rod 1080. It features a solid shaft 1082 with a formed handle 1084 at the proximal end and an interface structure 1086 at the distal end. The interface structure 1086 is formed to mate with the proximal end geometry 1012 of the random coil support device 1000 (see FIG. 59). FIG. 60 shows the random coil support device 1000 as it is deployed from the introducer tube 1070.

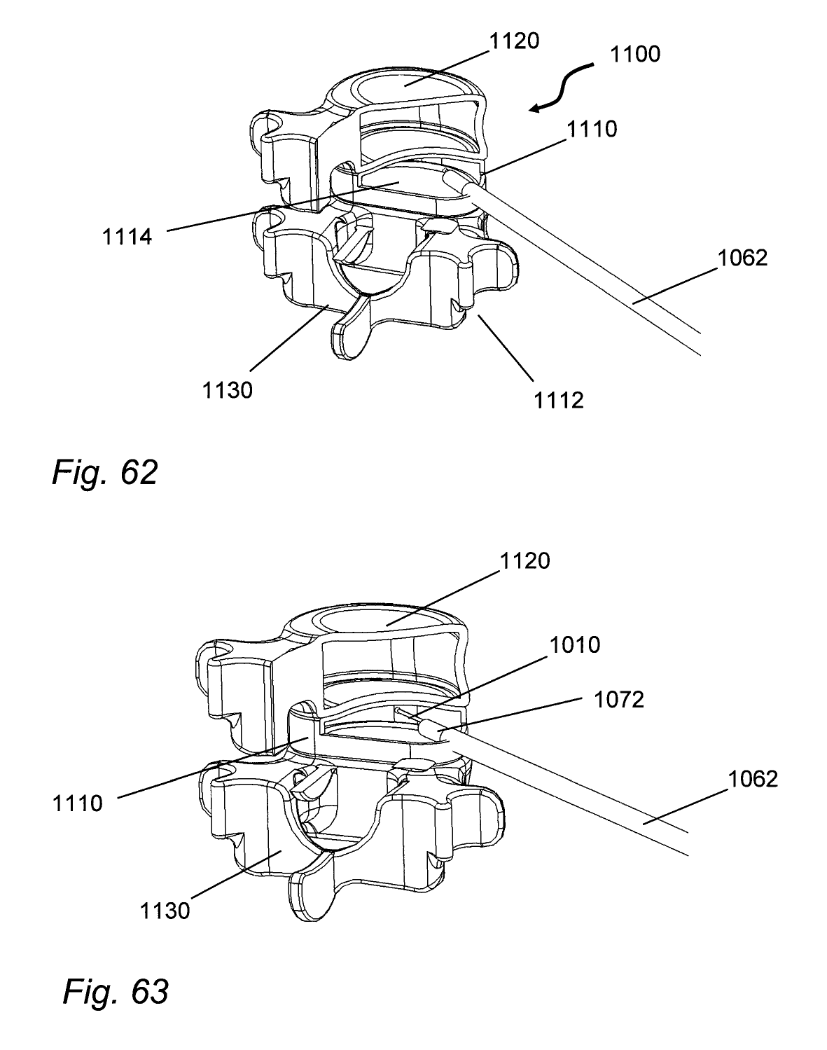

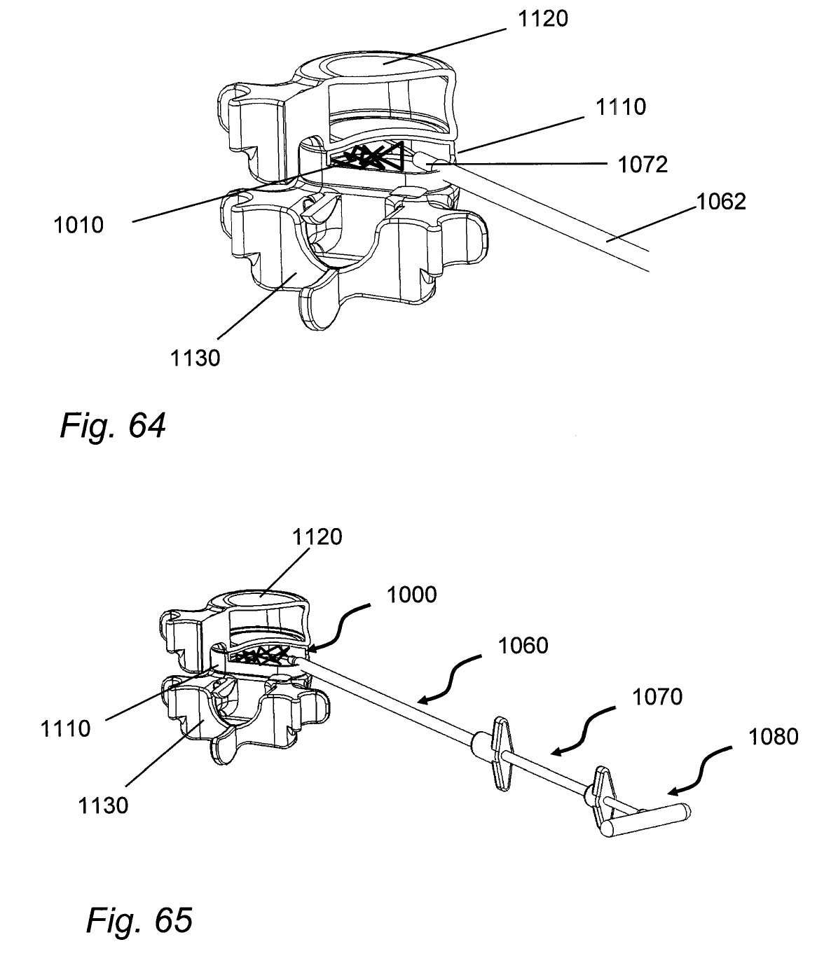

FIGS. 61 through 65 depict the random coil support device 1000 with its delivery tool 1050 positioned within the disk space 1114 of a vertebral disk 1110 situated between two vertebrae 1120 and 1130. In use, the delivery sheath 1060 is passed through the external tissue of the body and through a sized opening in the disk wall where it acts as a conduit for the rest of the delivery tool. The introducer tube 1070 with the random coil support device 1000, and deployment rod 1080 assembled within it is inserted through the delivery sheath 1060 until the distal end of the introducer 1070 is positioned at the desired location within the disk space 1114. FIGS. 63, 64, and 65 illustrate the deployment sequence for the random coil support device 1000. The handle 1084 of the deployment rod 1080 is pushed into the introducer tube 1070 forcing the distal end of the random coil support device 1000 out of the distal end of the introducer tube into the disk space 1114. The blunted end 1014 of the random coil support device 1000 will contact the inner wall of the disk 1110 and stop. Subsequent force created by the continued pushing on the deployment rod 1070 will cause the coil body 1010 of the random coil support device 1000 to buckle. The buckled section will move in a random direction until some portion of the coil body 1010 again contacts the inner wall of the disk 1110. This process is continued (i.e., buckling/contact with wall or other portions of the coil body/etc.) forming a randomized mesh of coil body 1010 within the disk space (see FIGS. 64 and 65). Depending on the size of the disk space volume and length of the random coil support device 1000, multiples of the devices may be used to completely fill the volume as desired. The combined, interwoven, meshed structure of the random coil support device 1000 effectively creates a support structure spanning the two vertebrae 1120 and 1130. This random coil device and its delivery system may also be used in a similar fashion for deployment within a vertebral body.

The random coil support device 1000 shown here is but one embodiment of the possible designs for a device of this type. Various wire cross sections can be envisioned along with different body configurations from a straight wire to one with multiple random kinks meant to help create the random buckling of the body during deployment. In addition to fixed lengths of the coil body, a continuous, coiled or wound length of coil body could be used with a delivery system that deploys the desired amount of continuous coil body into the volume, cutting to length (and forming the now proximal end of the wire) at the appropriate point.

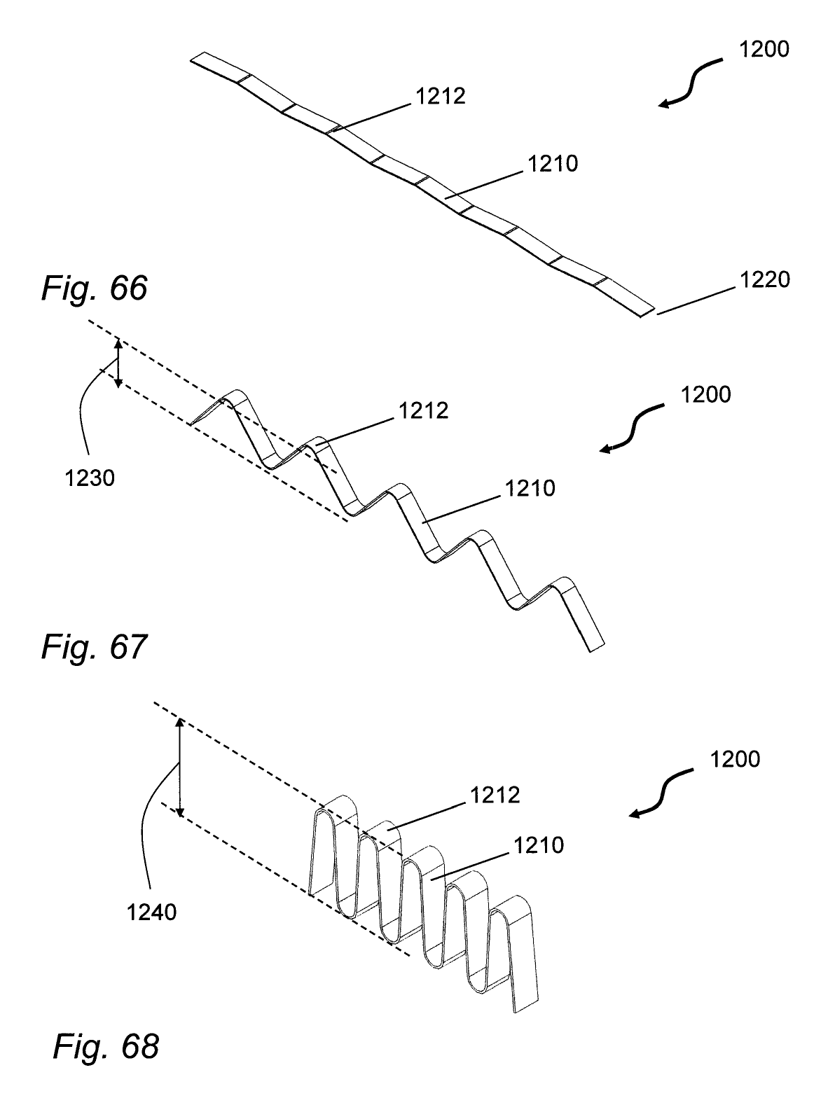

FIGS. 66, 67, and 68 depict a unique embodiment of the random coil support device here designated as a flexible coil 1200 that, by its design, applies a unidirectional force on the containment walls of the volume where it is deployed (i.e. disk space or within a vertebral body). The uniqueness of this design is in the thin rectangular cross section 1220 of the coil body 1210 and the preformed bends 1212 along its length. During deployment, the distal end of the coil body 1210 contacts a section of the containment volume and stops. As the deployment continues, the coil body 1210 buckles at the preformed bends 1212 creating a folded/accordion type structure. As the ends of the coil body 1210 are forced further together the folds come together causing the height of the folds 1230 to increase 1240 until the preformed bends 1212 contact the upper and lower walls of the containment volume. Additional pressure on the proximal end of the coil body forces the preformed bends 1212 into the upper and lower walls of the containment volume creating a separation (or holding) force between them. The wide cross section area 1220 spreads the separation force over a larger area.

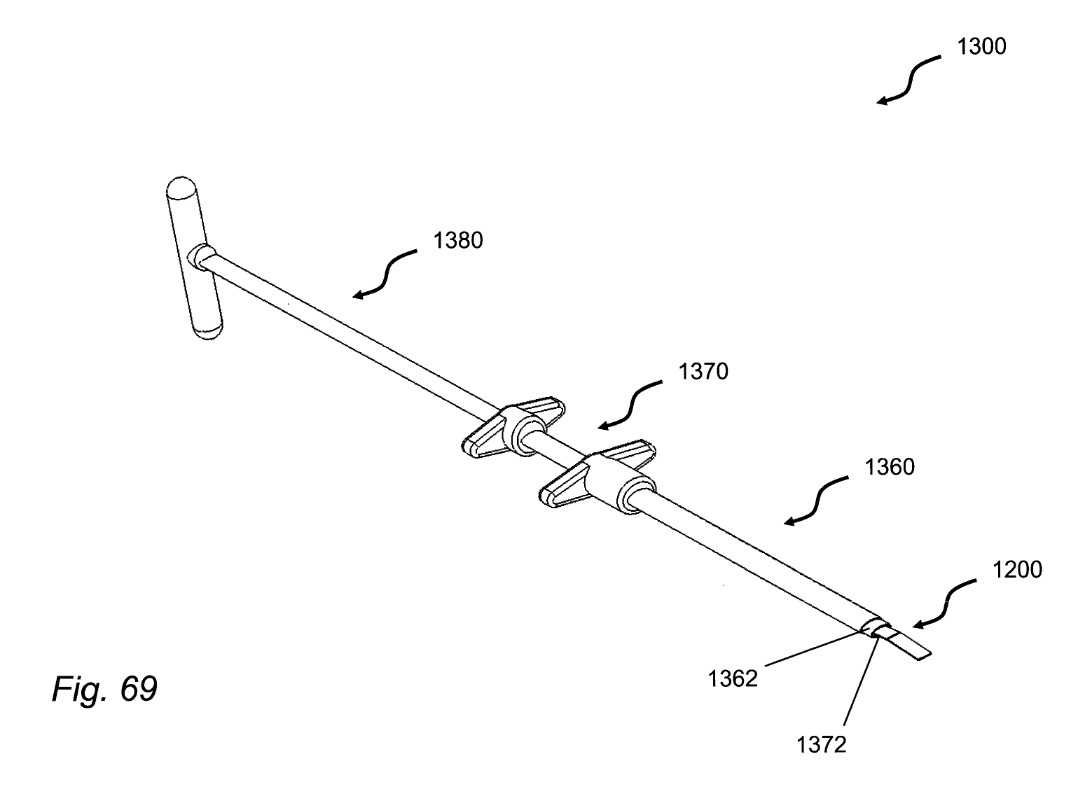

FIG. 69 show an delivery tool set 1300 fashioned for the flexible coil 1200 that is similar in design to the delivery tool set 1050 for the random coil 1000. The main difference is the shape or cross section of the bodies of the various components; delivery sheath 1360, introducer tube 1370, and deployment rod 1380. The cross section of the sheath 1362 and introducer tube 1372 shown here has a short height and long width to match the thin/wide rectangular cross section 1220 of the flexible coil 1200. This allows for a smaller dilated opening in the body tissue that the delivery sheath 1360 passes through.

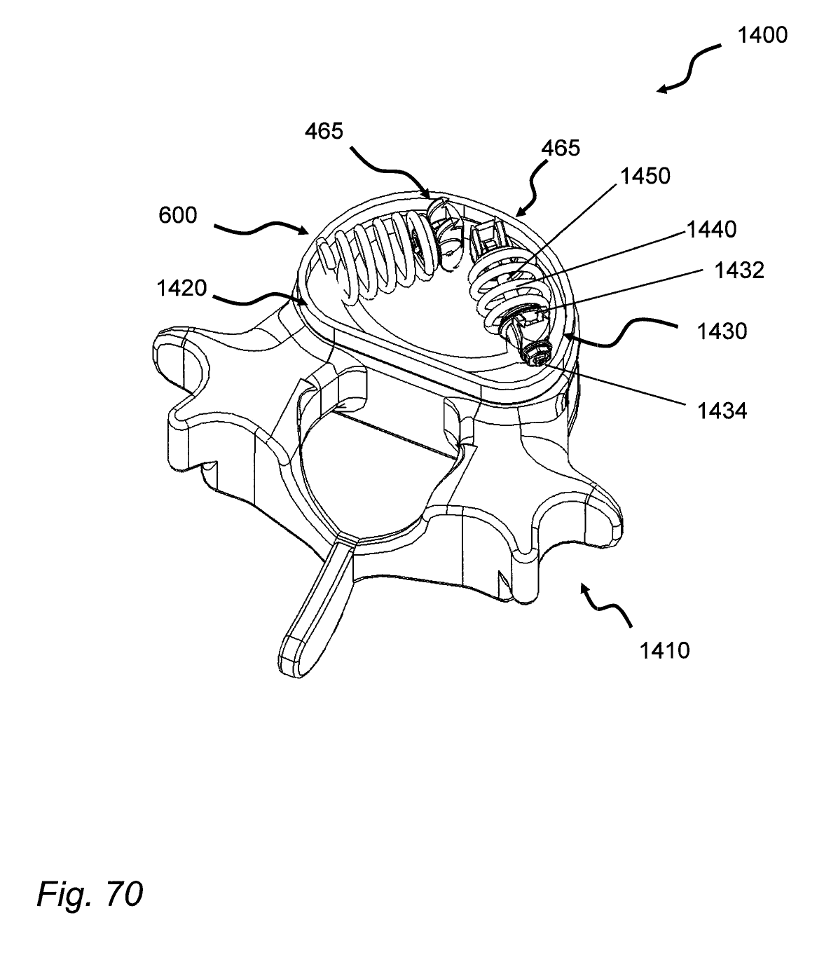

FIG. 70 illustrates the potential of combining 2 of the previously defined interbody fusion devices, expanding cam 465 and spring cage 600, in a single fusion procedure. This figure shows 2 different potential combinations. In the left configuration, an expanding cam 465 is first installed within the disk 1420 followed by a spring cage 600 whose distal end mechanically interfaces with a properly formed nut on the expanding cam 465. In the right configuration, an expanding cam 465 is first installed within the disk 1420 followed by a shortened version of spring 600 (labeled 1440). The rod 1450 that was used to guide the nut for expanding cam 465 remains in place guiding a washer 1432 against the proximal end of the spring 1440. A slightly altered version of expanding cam 465 generally labeled 1430 installs over rod 1450, it followed by a nut 1434 that is used to expand the cams as it is threaded over rod 1450. The orientation of the cams is in the opposite direction as those of the first expanding cam 465 thus capturing the spring 1440 between spikes embedded in an opposing fashion. Other combinations of the different devices depicted in this document are possible.

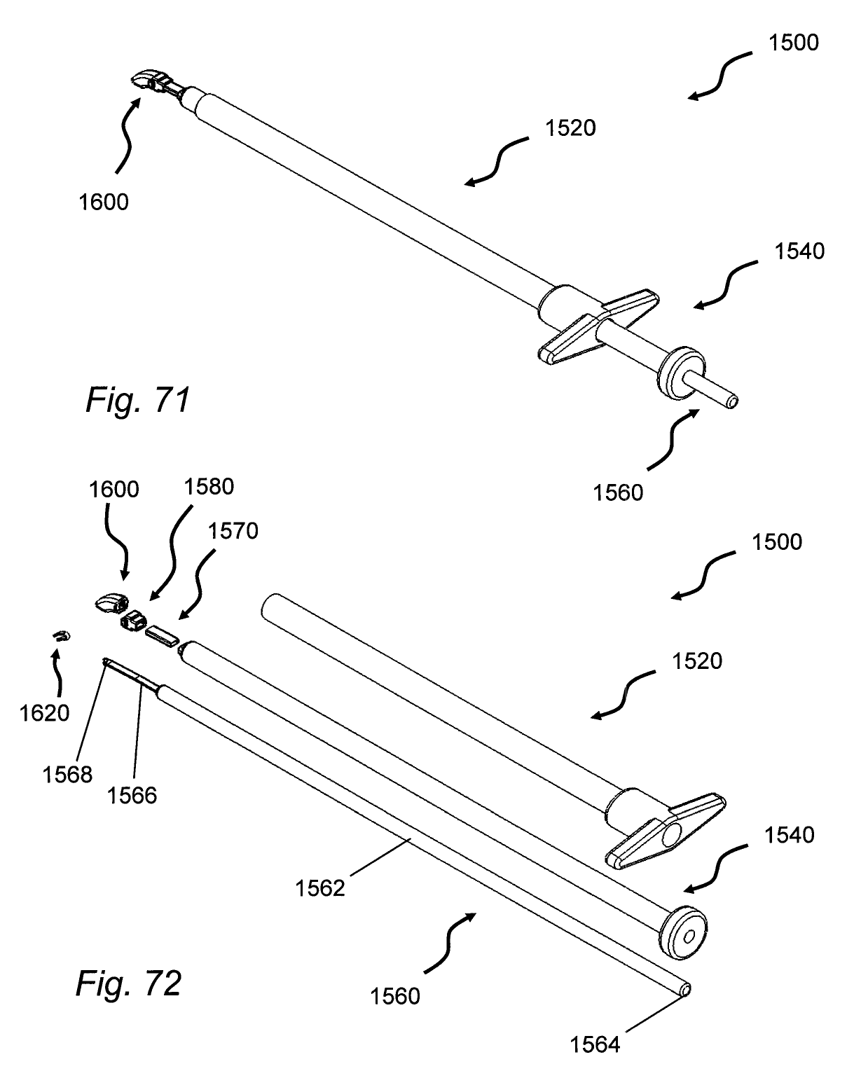

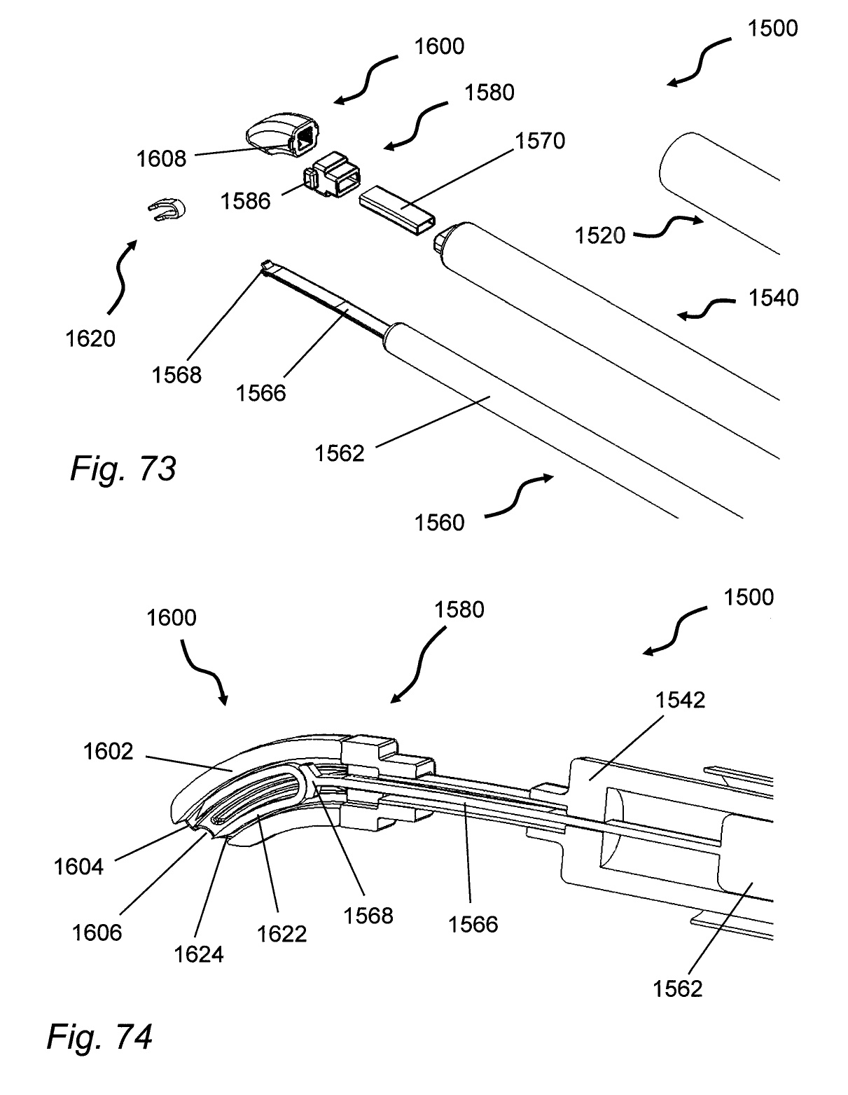

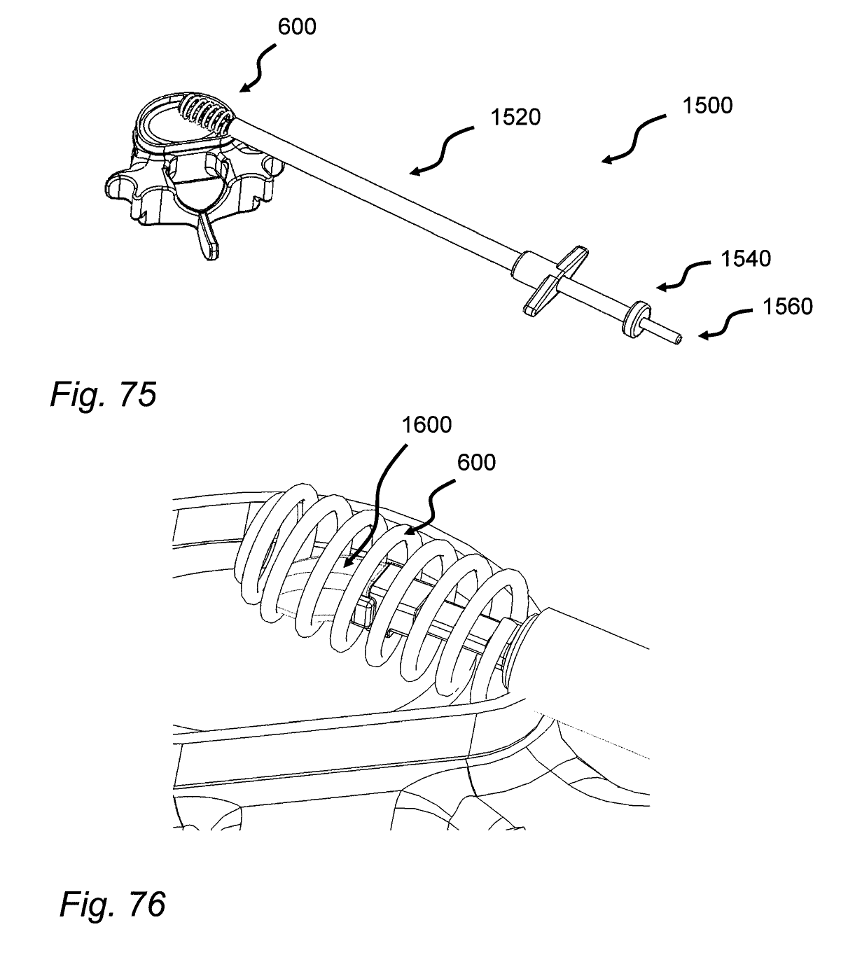

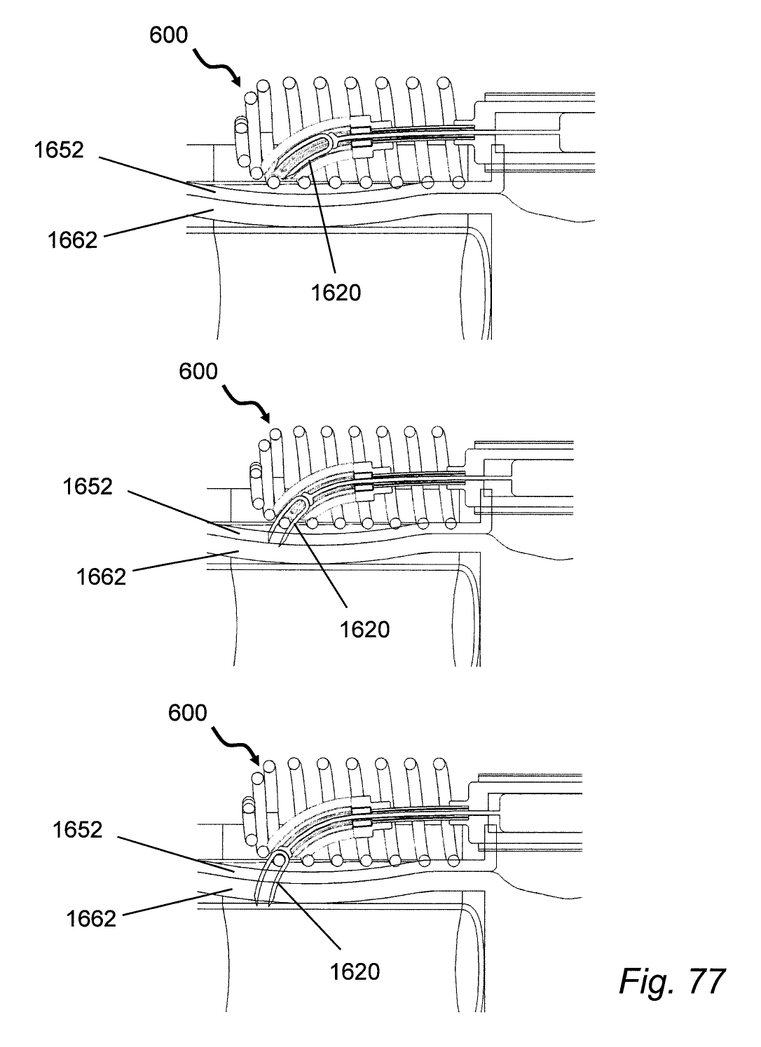

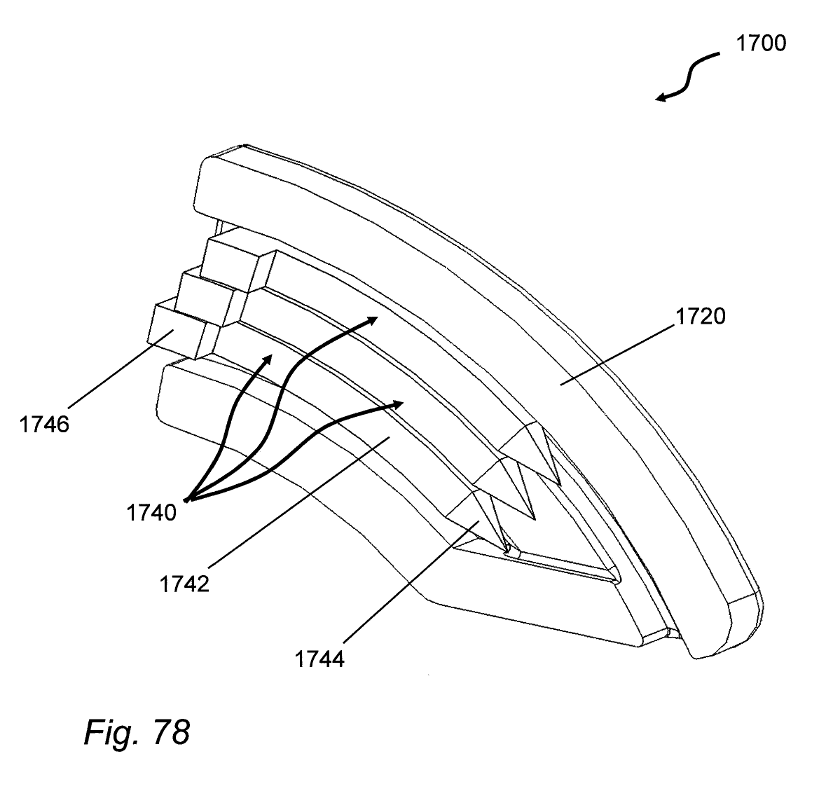

FIGS. 71 through 77 show an embodiment of a stapling tool, generally designated 1500 used to anchor a spring cage 600 to the two vertebrae on either side of the disk in which it was deployed. Referring to FIGS. 71, 72, 73, and 74; the stapling tool consists a guide body assembly 1540, a ram 1560, a cartridge 1600 and the anchoring/fixation device, shown here as a staple 1620. It should be noted that the stapling tool is not limited to the use of staples, and that other types of anchoring/fixation devices, such as brads or nails, can be used with the stapling tool of the invention. The stapler 1500 would be inserted through the delivery sheath 1520 which was installed in the disk and used to deploy the spring cage 600 (see FIGS. 75 and 76). The guide body assembly 1540 is an assembly of the rigid guide body 1542, the flexible guide 1570, and the cartridge adapter 1580. The flexibility of the flexible guide 1570, which is curved to direct the cartridge 1600 radially make contact with the larger ID spring cage 600, allows the distal end of the guide body assembly 1540 to deflect during insertion and removal to fit within the delivery sheath 1520. Installed within the guide body assembly 1540 is the ram 1560. The ram 1560 has a solid cylindrical body 1562 with a strike point 1564 on the proximal end and a hammer end 1568 connected via a flexible beam 1566. The ram 1560 slides within the guide body assembly 1540. The distal end of the cartridge adapter 1580 has locking ears 1586 that locate and contain the tabs 1608 on the cartridge 1600. Within the body 1602 of the cartridge 1600 is a cavity that contains ribs 1604 that constrain and guide the staple 1620 in addition to guiding the hammer 1568 end of the ram 1560. A notch 1606 on the distal end of the cartridge 1600 is used to position in over the wire coil of the spring cage 600 so that the staple 1620 captures the wire coil as it embeds in the vertebral bone. The staple 1620 has a curved body 1622 with two legs that end in sharp, angled points 1624. The cross section of the staple body 1622 can be of various shapes (rectangular, circular, etc.) and may contain barbs or the like to help contain it in the bone after deployment. FIG. 77 illustrates the deployment of the staple 1620 into the upper plate 1652 of a vertebral body 1662. When the strike point 1564 of the ram 1560 is struck with either a single hard blow or a high repetition of lighter blows (i.e. impact hammer) it transfers the force through the ram cylindrical body 1562, the flexible beam 1566, and through the hammer end 1568 to the head of the staple 1620 driving it down the cartridge guide path over the spring cage 600 wire coil into the bone. Multiple staples 1620 would be used to anchor the spring cage 600 to both the upper and lower vertebral bodies. The energy for the blows that deploy the staples can be delivered by various means; manually with a hammer, using a powered (pneumatic, electric, etc.) ram to single hard blow, or a powered impact hammer type device that generates a high repetition of less energetic blows. Various configurations of the cartridge (single use, reloadable, multiple staples) is possible. FIG. 78 shows a curved nail version of the stapling tool cartridge generally designated 1700. The cartridge 1720 contains one or more of the curved nails 1740 which consist of a thin curved body 1742, a penetrating point 1744, and a head 1746. The nails 1740 are deployed using a similar ram device as shown in stapling tool 1500. This embodiment shows 3 nails in the cartridge which would be deployed sequentially. Additional structural features such as barbs or different head designs are possible while retaining the basic curved shape that allows the nail to be deployed off axis to the delivery tool.

While the inventions have been illustrated and described in detail in the drawings and foregoing description, the same is to be considered as illustrative and not restrictive in character, it being understood that the embodiments have been shown and described and that all changes and modifications that come within the spirit of these inventions are desired to be protected.

What is claimed is:

1. A rotating interbody spinal fusion device comprising:
a hollow cam shaped cylindrical body with base planar surfaces connected to expanded planar surfaces via cam surfaces;
a plurality of fenestrations along the cam body, the fenestrations extending from the exterior to the interior of the body; and
a delivery tool engagement feature for rotating the cam body.

2. The device of claim 1, further comprising one or more protrusions extending from the cam body, the protrusions having pointed ends.

3. The device of claim 1, wherein the cam body is tapered along the length of the body.

4. The device of claim 1 further comprising a second hollow cam shaped cylindrical body linked to the first cam body via a swivel joint to allow the first and second cams to rotate relative to each other, the second hollow cam shaped body comprising base planar surfaces connected to expanded planar surfaces via cam surfaces, a plurality of fenestrations along the length of the cam body extending from the exterior to the interior of the body, and a delivery tool engagement feature for rotating the cam body.

5. The device of claim 4, further comprising one or more protrusions extending from the first and second cam bodies, the protrusions having pointed ends, wherein the one or more protrusions extend from the second cam body in an opposite direction to the one or more protrusions extending from the first cam body.

6. The device of claim 4, wherein the first and second cam bodies are tapered along the length of the bodies.

7. The device of claim 1, wherein the device is cannulated for insertion over a guide pin or a guide wire.

8. An expanding intervertebral device comprising:
two cams, each cam comprising two pin holes, a cam surface and one or more protrusions extending from cam surfaces, the protrusions having pointed ends;
an anchor rod comprising a mating hole and a threaded surface opposite the mating hole;
a pivot pin; and
a locking nut comprising an integral washer and an interior threaded surface;
wherein the pin holes of each cam are coupled to the mating hole of the anchor rod via the pivot pin and anchor rod is coupled to the locking nut via their threaded surfaces, and wherein the cams are rotated 180 degrees relative to each other when assembled.

9. The device of claim 8, wherein the device is cannulated for insertion over a guide pin or a guide wire.

10. An intervertebral device comprising a helical spring body having an inner and an outer diameter, a cross section diameter, a defined pitch length, and a defined number of turns.

11. The device of claim 10, wherein the spring body tapers along the length of the body.

12. The device of claim 10, wherein the inner and outer diameters are uniform along the length of the spring body.

13. The device of claim 10, wherein the inner and outer diameters are variable along the length of the spring body.

14. The device of claim 10, wherein the cross section diameter is non-circular.

15. The device of claim 10, further comprising a second helical spring body disposed within the first spring body, wherein the outer diameter of the second spring body is larger than the inner diameter of the first spring body.

16. The device of claim 15, wherein the second spring body has an opposite hand than the first spring body.

17. The device of claim 10, further comprising an expandable, cylindrical shaped containment cage comprising two side walls having a proximal and a distal end and multiple perforations, end plates at the distal end of the side walls, and a plurality of bridging arms connecting the side walls.

18. The device of claim 10, wherein the device is cannulated for insertion over a guide pin or a guide wire.

19. An intervertebral device comprising a coil body having a defined length, a cross section diameter, a distal end and a proximal end, the distal end having a blunted shape, wherein the coil body is adapted to buckle along the length of the body when force is applied against the ends of the coil.

20. The device of claim 19, wherein the coil body randomly buckles along the length of the coil body when force is applied against the ends of the coil

21. The device of claim 19, wherein the coil body has a rectangular cross section and a plurality of preformed bends along the length of the body where the coil body buckles when force is applied against the ends of the coil.

22. The device of claim 19, wherein the device is cannulated for insertion over a guide pin or a guide wire.
